SEQUENCE LISTING (189 sequences)

SEQ ID NO: 1 - DNA, 25 bp, Artificial
  Synthetic
tccctacacgacgctcttccgatct

SEQ ID NO: 2 - DNA, 25 bp, Artificial
  Synthetic
agttcagacgtgtgctcttccgatc

SEQ ID NO: 3 - DNA, 16 bp, Artificial
  Synthetic
tccctacacgacgctc

SEQ ID NO: 4 - DNA, 9 bp, Artificial
  Synthetic
ttccgatct

SEQ ID NO: 5 - DNA, 17 bp, Artificial
  Synthetic
agttcagacgtgtgctc

SEQ ID NO: 6 - DNA, 8 bp, Artificial
  Synthetic
ttccgatc

SEQ ID NO: 7 - DNA, 49 bp, Artificial
  Synthetic
aatgatacggcgaccaccgagatctacactctttccctacacgacgctc

SEQ ID NO: 8 - DNA, 55 bp, Artificial
  Synthetic
  misc_feature (25)..(30): n is a, c, g, or t
caagcagaagacggcatacgagatnnnnnngtgactggagttcagacgtgtgctc

SEQ ID NO: 9 - DNA, 49 bp, Artificial
  Synthetic
caagcagaagacggcatacgagatgtgactggagttcagacgtgtgctc

SEQ ID NO: 10 - DNA, 33 bp, Artificial
  Synthetic
aatgatacggcgaccaccgagatctacactctt

SEQ ID NO: 11 - DNA, 32 bp, Artificial
  Synthetic
caagcagaagacggcatacgagatgtgactgg

SEQ ID NO: 12 - DNA, 38 bp, Artificial
  Synthetic
  misc_feature (25)..(30): n is a, c, g, or t
caagcagaagacggcatacgagatnnnnnngtgactgg

SEQ ID NO: 13 - DNA, 24 bp, Artificial
  Synthetic
caagcagaagacggcatacgagat

SEQ ID NO: 14 - DNA, 58 bp, Artificial
  Synthetic
aatgatacggcgaccaccgagatctacactctttccctacacgacgctcttccgatct

SEQ ID NO: 15 - DNA, 57 bp, Artificial
  Synthetic
caagcagaagacggcatacgagatgtgactggagttcagacgtgtgctcttccgatc

SEQ ID NO: 16 - DNA, 63 bp, Artificial
  Synthetic
  misc_feature (25)..(30): n is a, c, g, or t
caagcagaagacggcatacgagatnnnnnngtgactggagttcagacgtgtgctcttccgatc

SEQ ID NO: 17 - DNA, 8 bp, Artificial
  Synthetic
ttccgatc

SEQ ID NO: 18 - DNA, 21 bp, Homo Sapiens
gtatgattttttgggggttag

SEQ ID NO: 19 - DNA, 25 bp, Homo Sapiens
cttaacaccaacctactaaactaaa

SEQ ID NO: 20 - DNA, 23 bp, Homo Sapiens
tggatagatatggaagtatgtga

SEQ ID NO: 21 - DNA, 21 bp, Homo Sapiens
aaaaaaccctcatacactcaa

SEQ ID NO: 22 - DNA, 25 bp, Homo Sapiens
ttaaatgtaaagtaatgtttgtttt

SEQ ID NO: 23 - DNA, 26 bp, Homo Sapiens
aaattcacaaactctaactactacac

SEQ ID NO: 24 - DNA, 24 bp, Homo Sapiens
ggttagatttttatggtagtgagg

SEQ ID NO: 25 - DNA, 26 bp, Homo Sapiens
aaactattaactaacactaaactccc

SEQ ID NO: 26 - DNA, 25 bp, Homo Sapiens
ttagtttttttagtatgattttgag

SEQ ID NO: 27 - DNA, 23 bp, Homo Sapiens
caccacaaaaatcacaatactat

SEQ ID NO: 28 - DNA, 25 bp, Homo Sapiens
gttaagaaattaataggaaaaagaa

SEQ ID NO: 29 - DNA, 20 bp, Homo Sapiens
aaaaccccatattacttccc

SEQ ID NO: 30 - DNA, 26 bp, Homo Sapiens
tgtgaatagaaaaagttttaaatatg

SEQ ID NO: 31 - DNA, 21 bp, Homo Sapiens
caaaccctccaccctaacttc

SEQ ID NO: 32 - DNA, 25 bp, Homo Sapiens
tatatgtgtgtaggttgaataaaat

SEQ ID NO: 33 - DNA, 25 bp, Homo Sapiens
tccatttcatatcaatactaatatt

SEQ ID NO: 34 - DNA, 20 bp, Homo Sapiens
taggagtaggaatggggagg

SEQ ID NO: 35 - DNA, 24 bp, Homo Sapiens
ctcaataatacttctcctacccac

SEQ ID NO: 36 - DNA, 22 bp, Homo Sapiens
gttttttgtttttgtaggttga

SEQ ID NO: 37 - DNA, 24 bp, Homo Sapiens
cttcaaaatacaaaacactcatct

SEQ ID NO: 38 - DNA, 23 bp, Homo Sapiens
gaggaggagtaggagtagatgtt

SEQ ID NO: 39 - DNA, 23 bp, Homo Sapiens
gaggaggagtaggagtagatgtt

SEQ ID NO: 40 - DNA, 22 bp, Homo Sapiens
gtttgttttgagatgtgagaat

SEQ ID NO: 41 - DNA, 23 bp, Homo Sapiens
ataacatccttacaaactcacaa

SEQ ID NO: 42 - DNA, 27 bp, Homo Sapiens
gttagaaggtatagaaataattgttat

SEQ ID NO: 43 - DNA, 21 bp, Homo Sapiens
tctccaaactctaaaaaccct

SEQ ID NO: 44 - DNA, 26 bp, Homo Sapiens
aggttgtatgttgagttagatgttat

SEQ ID NO: 45 - DNA, 28 bp, Homo Sapiens
ctatataaccatactattaaccaacatt

SEQ ID NO: 46 - DNA, 26 bp, Homo Sapiens
gtttggtaatttatttagagaagtaa

SEQ ID NO: 47 - DNA, 22 bp, Homo Sapiens
cccacaaataaaaaaaatactc

SEQ ID NO: 48 - DNA, 25 bp, Homo Sapiens
aagagtattaggaaaagtgtaggtt

SEQ ID NO: 49 - DNA, 22 bp, Homo Sapiens
tatccaaaaaaacaaaataacc

SEQ ID NO: 50 - DNA, 27 bp, Homo Sapiens
gtgttgttatttaagttattgagagta

SEQ ID NO: 51 - DNA, 24 bp, Homo Sapiens
aaatacctcaaaaaacctaaacta

SEQ ID NO: 52 - DNA, 22 bp, Homo Sapiens
ttggtaggtttggagttgatag

SEQ ID NO: 53 - DNA, 22 bp, Homo Sapiens
aaaaataaacactaaaatcccc

SEQ ID NO: 54 - DNA, 27 bp, Homo Sapiens
ttatatttatttttaattatatagggg

SEQ ID NO: 55 - DNA, 24 bp, Homo Sapiens
ggttttctgcttcctttcaaccag

SEQ ID NO: 56 - DNA, 21 bp, Homo Sapiens
ttgtagtgtttagtggagggg

SEQ ID NO: 57 - DNA, 21 bp, Homo Sapiens
ctacttcaccttcaaccccta

SEQ ID NO: 58 - DNA, 22 bp, Homo Sapiens
ttaggtttggattttaattttg

SEQ ID NO: 59 - DNA, 21 bp, Homo Sapiens
ccctaacccttatctactcca

SEQ ID NO: 60 - DNA, 22 bp, Homo Sapiens
tgggttttgttgttatagtttt

SEQ ID NO: 61 - DNA, 23 bp, Homo Sapiens
atatctaccctcttctcttcctc

SEQ ID NO: 62 - DNA, 29 bp, Homo Sapiens
gttattagtttttattgtagttattttga

SEQ ID NO: 63 - DNA, 24 bp, Homo Sapiens
aataacaaacaaacacacaaaaca

SEQ ID NO: 64 - DNA, 21 bp, Homo Sapiens
tgggtgttgttattttgttga

SEQ ID NO: 65 - DNA, 21 bp, Homo Sapiens
ctacaaaaatacacaccccaa

SEQ ID NO: 66 - DNA, 20 bp, Homo Sapiens
ttgttggttttttggggatt

SEQ ID NO: 67 - DNA, 22 bp, Homo Sapiens
accctacaaatcctctacctcc

SEQ ID NO: 68 - DNA, 24 bp, Homo Sapiens
gtttattttgtaggttttttgttt

SEQ ID NO: 69 - DNA, 24 bp, Homo Sapiens
ctactaaccctctaaaaacctaac

SEQ ID NO: 70 - DNA, 23 bp, Homo Sapiens
ttaagtttgaaggtgtaggtttt

SEQ ID NO: 71 - DNA, 21 bp, Homo Sapiens
aaaacccacacacacacataa

SEQ ID NO: 72 - DNA, 25 bp, Homo Sapiens
atagtgaagatgttagtttgttttt

SEQ ID NO: 73 - DNA, 24 bp, Homo Sapiens
aacacacttacctaataaccaaac

SEQ ID NO: 74 - DNA, 27 bp, Homo Sapiens
gttagaaggtatagaaataattgttat

SEQ ID NO: 75 - DNA, 21 bp, Homo Sapiens
tctccaaactctaaaaaccct

SEQ ID NO: 76 - DNA, 24 bp, Homo Sapiens
ggtttagtagttaggatagggtag

SEQ ID NO: 77 - DNA, 25 bp, Homo Sapiens
aaaaataaccaaacctaaactatac

SEQ ID NO: 78 - DNA, 25 bp, Homo Sapiens
taggtttttttagtatagtatggtg

SEQ ID NO: 79 - DNA, 26 bp, Homo Sapiens
caactcctaacttactaatactaacc

SEQ ID NO: 80 - DNA, 18 bp, Homo Sapiens
ggaggttgggagaggttt

SEQ ID NO: 81 - DNA, 26 bp, Homo Sapiens
ccaactacaaacatctactttaatac

SEQ ID NO: 82 - DNA, 23 bp, Homo Sapiens
gttgtaggtgtttattggtattg

SEQ ID NO: 83 - DNA, 23 bp, Homo Sapiens
cctaaataaacaactccaaataa

SEQ ID NO: 84 - DNA, 30 bp, Homo Sapiens
ttagttttttagtttatagttagtattagt

SEQ ID NO: 85 - DNA, 23 bp, Homo Sapiens
tctcatccaatacaaaaaaaata

SEQ ID NO: 86 - DNA, 20 bp, Homo Sapiens
gggtaggaggagttgttggt

SEQ ID NO: 87 - DNA, 22 bp, Homo Sapiens
ataccccccatatcacaactac

SEQ ID NO: 88 - DNA, 25 bp, Homo Sapiens
tttgttaggttaagtaatttgtaaa

SEQ ID NO: 89 - DNA, 25 bp, Homo Sapiens
catctcctacttaaataacttcaat

SEQ ID NO: 90 - DNA, 24 bp, Homo Sapiens
ggaggttgtagtgagttaagatta

SEQ ID NO: 91 - DNA, 25 bp, Homo Sapiens
tcaacaactactcaattacacacta

SEQ ID NO: 92 - DNA, 21 bp, Homo Sapiens
attttttttggttggattgtt

SEQ ID NO: 93 - DNA, 20 bp, Homo Sapiens
tcacaaacacacaaacccaa

SEQ ID NO: 94 - DNA, 25 bp, Homo Sapiens
ttttaagaagtttttgtgttattat

SEQ ID NO: 95 - DNA, 24 bp, Homo Sapiens
tctaaaaatacctaaatacaaacc

SEQ ID NO: 96 - DNA, 25 bp, Homo Sapiens
atggagtttgagttatataggtatg

SEQ ID NO: 97 - DNA, 24 bp, Homo Sapiens
aaactcctaaactcaaaaaatcta

SEQ ID NO: 98 - DNA, 27 bp, Homo Sapiens
tttattttatatttaagtttaggtgat

SEQ ID NO: 99 - DNA, 26 bp, Homo Sapiens
cccacttacatatatataaacctaaa

SEQ ID NO: 100 - DNA, 23 bp, Homo Sapiens
ggggtattttttattattttatt

SEQ ID NO: 101 - DNA, 22 bp, Homo Sapiens
acacacaacttcaaaaacttca

SEQ ID NO: 102 - DNA, 23 bp, Homo Sapiens
gaaagaggggataataaatagtt

SEQ ID NO: 103 - DNA, 25 bp, Homo Sapiens
cttattactatcactcccaaactaa

SEQ ID NO: 104 - DNA, 23 bp, Homo Sapiens
ggtgttataggtagggtagagaa

SEQ ID NO: 105 - DNA, 22 bp, Homo Sapiens
ccaacatttatcattttcttca

SEQ ID NO: 106 - DNA, 19 bp, Homo Sapiens
ggttttgggaggttagtgg

SEQ ID NO: 107 - DNA, 20 bp, Homo Sapiens
tcaccaaaactccaacttca

SEQ ID NO: 108 - DNA, 22 bp, Homo Sapiens
gagaaggtaatgttgttggaat

SEQ ID NO: 109 - DNA, 24 bp, Homo Sapiens
ataacacaaacattaacttcaaac

SEQ ID NO: 110 - DNA, 29 bp, Homo Sapiens
ttttatgtatagattaatagtaaagtttt

SEQ ID NO: 111 - DNA, 25 bp, Homo Sapiens
aaaccaacatttctctataactact

SEQ ID NO: 112 - DNA, 24 bp, Homo Sapiens
aggttgtttttttattttttagat

SEQ ID NO: 113 - DNA, 22 bp, Homo Sapiens
tttccctccctttaataactat

SEQ ID NO: 114 - DNA, 21 bp, Homo Sapiens
agtatttttattgggttggat

SEQ ID NO: 115 - DNA, 24 bp, Homo Sapiens
cctactacctcaaattaactaaaa

SEQ ID NO: 116 - DNA, 26 bp, Homo Sapiens
agtagatgtattattgtgaagaagaa

SEQ ID NO: 117 - DNA, 26 bp, Homo Sapiens
aacaataaataataaatctctcaaaa

SEQ ID NO: 118 - DNA, 22 bp, Homo Sapiens
tgaaggaaatgagagtaaaggt

SEQ ID NO: 119 - DNA, 20 bp, Homo Sapiens
cccttctccctaaaaaaaac

SEQ ID NO: 120 - DNA, 22 bp, Homo Sapiens
ttttaaagaagtttttatgggt

SEQ ID NO: 121 - DNA, 23 bp, Homo Sapiens
ataaaccaaacaacactacacat

SEQ ID NO: 122 - DNA, 26 bp, Homo Sapiens
tttagtgttagaattgaaagagtaga

SEQ ID NO: 123 - DNA, 26 bp, Homo Sapiens
ttaaccttaactatatctaacaaaaa

SEQ ID NO: 124 - DNA, 22 bp, Homo Sapiens
ggtattttgaagaggtaggttt

SEQ ID NO: 125 - DNA, 21 bp, Homo Sapiens
acctaaataccccaaactcat

SEQ ID NO: 126 - DNA, 21 bp, Homo Sapiens
atttgattttgtggtagtgga

SEQ ID NO: 127 - DNA, 21 bp, Homo Sapiens
aaaatccccacctctacttaa

SEQ ID NO: 128 - DNA, 23 bp, Homo Sapiens
gttttttgttttagaattggtta

SEQ ID NO: 129 - DNA, 22 bp, Homo Sapiens
aatccataaataaaacaaaccc

SEQ ID NO: 130 - DNA, 28 bp, Homo Sapiens
gtttttttatatataattataagttgtt

SEQ ID NO: 131 - DNA, 26 bp, Homo Sapiens
ataaaaatacttactactacctttcc

SEQ ID NO: 132 - DNA, 21 bp, Homo sapiens
tttggttttggtgtatttttt

SEQ ID NO: 133 - DNA, 27 bp, Homo sapiens
caaaacctactataatacttctaaact

SEQ ID NO: 134 - DNA, 23 bp, Homo sapiens
gtggggtaagagtttttagtatt

SEQ ID NO: 135 - DNA, 23 bp, Homo sapiens
ccaatatatctatacaaatcccc

SEQ ID NO: 136 - DNA, 21 bp, Homo sapiens
ggttgagtttattgttttggg

SEQ ID NO: 137 - DNA, 23 bp, Homo sapiens
tttaccaacaacaacaacctaac

SEQ ID NO: 138 - DNA, 16 bp, Artificial
  Synthetic
atgggcagtcggtgat

SEQ ID NO: 139 - DNA, 15 bp, Artificial
  Synthetic
atgggcagtcggtga

SEQ ID NO: 140 - DNA, 19 bp, Artificial
  Synthetic
tctatgggcagtcggtgat

SEQ ID NO: 141 - DNA, 15 bp, Artificial
  Synthetic
tctatgggcagtcgg

SEQ ID NO: 142 - DNA, 16 bp, Artificial
  Synthetic
tctatgggcagtcggt

SEQ ID NO: 143 - DNA, 17 bp, Artificial
  Synthetic
tctatgggcagtcggtg

SEQ ID NO: 144 - DNA, 18 bp, Artificial
  Synthetic
tctatgggcagtcggtga

SEQ ID NO: 145 - DNA, 14 bp, Artificial
  Synthetic
gggcagtcggtgat

SEQ ID NO: 146 - DNA, 14 bp, Artificial
  Synthetic
tgtctccgactcag

SEQ ID NO: 147 - DNA, 13 bp, Artificial
  Synthetic
tgtctccgactca

SEQ ID NO: 148 - DNA, 18 bp, Artificial
  Synthetic
tgcgtgtctccgactcag

SEQ ID NO: 149 - DNA, 15 bp, Artificial
  Synthetic
tgcgtgtctccgact

SEQ ID NO: 150 - DNA, 16 bp, Artificial
  Synthetic
tgcgtgtctccgactc

SEQ ID NO: 151 - DNA, 17 bp, Artificial
  Synthetic
tgcgtgtctccgactca

SEQ ID NO: 152 - DNA, 17 bp, Artificial
  Synthetic
gcgtgtctccgactcag

SEQ ID NO: 153 - DNA, 14 bp, Artificial
  Synthetic
gcgtgtctccgact

SEQ ID NO: 154 - DNA, 15 bp, Artificial
  Synthetic
gcgtgtctccgactc

SEQ ID NO: 155 - DNA, 16 bp, Artificial
  Synthetic
gcgtgtctccgactca

SEQ ID NO: 156 - DNA, 16 bp, Artificial
  Synthetic
cgtgtctccgactcag

SEQ ID NO: 157 - DNA, 13 bp, Artificial
  Synthetic
cgtgtctccgact

SEQ ID NO: 158 - DNA, 14 bp, Artificial
  Synthetic
cgtgtctccgactc

SEQ ID NO: 159 - DNA, 15 bp, Artificial
  Synthetic
cgtgtctccgactca

SEQ ID NO: 160 - DNA, 15 bp, Artificial
  Synthetic
gtgtctccgactcag

SEQ ID NO: 161 - DNA, 12 bp, Artificial
  Synthetic
gtgtctccgact

SEQ ID NO: 162 - DNA, 13 bp, Artificial
  Synthetic
gtgtctccgactc

SEQ ID NO: 163 - DNA, 14 bp, Artificial
  Synthetic
gtgtctccgactca

SEQ ID NO: 164 - DNA, 25 bp, Artificial
  Synthetic
ccactacgcctccgctttcctctct

SEQ ID NO: 165 - DNA, 16 bp, Artificial
  Synthetic
ccatctcatccctgcg

SEQ ID NO: 166 - DNA, 41 bp, Artificial
  Synthetic
ccactacgcctccgctttcctctctatgggcagtcggtgat

SEQ ID NO: 167 - DNA, 30 bp, Artificial
  Synthetic
ccatctcatccctgcgtgtctccgactcag

SEQ ID NO: 168 - DNA, 40 bp, Artificial
  Synthetic
ccactacgcctccgctttcctctctatgggcagtcggtga

SEQ ID NO: 169 - DNA, 29 bp, Artificial
  Synthetic
ccatctcatccctgcgtgtctccgactca

SEQ ID NO: 170 - DNA, 39 bp, Artificial
  Synthetic
ccactacgcctccgctttcctctctatgggcagtcggtg

SEQ ID NO: 171 - DNA, 28 bp, Artificial
  Synthetic
ccatctcatccctgcgtgtctccgactc

SEQ ID NO: 172 - DNA, 38 bp, Artificial
  Synthetic
ccactacgcctccgctttcctctctatgggcagtcggt

SEQ ID NO: 173 - DNA, 27 bp, Artificial
  Synthetic
ccatctcatccctgcgtgtctccgact

SEQ ID NO: 174 - DNA, 37 bp, Artificial
  Synthetic
ccactacgcctccgctttcctctctatgggcagtcgg

SEQ ID NO: 175 - DNA, 26 bp, Artificial
  Synthetic
ccatctcatccctgcgtgtctccgac

SEQ ID NO: 176 - DNA, 27 bp, Artificial
  Synthetic
ccactacgcctccgctttcctctctat

SEQ ID NO: 177 - DNA, 25 bp, Artificial
  Synthetic
ccactacgcctccgctttcctctct

SEQ ID NO: 178 - DNA, 24 bp, Artificial
  Synthetic
ccactacgcctccgctttcctctc

SEQ ID NO: 179 - DNA, 23 bp, Artificial
  Synthetic
ccactacgcctccgctttcctct

SEQ ID NO: 180 - DNA, 22 bp, Artificial
  Synthetic
ccactacgcctccgctttcctc

SEQ ID NO: 181 - DNA, 12 bp, Artificial
  Synthetic
ccatctcatccc

SEQ ID NO: 182 - DNA, 13 bp, Artificial
  Synthetic
ccatctcatccct

SEQ ID NO: 183 - DNA, 14 bp, Artificial
  Synthetic
ccatctcatccctg

SEQ ID NO: 184 - DNA, 15 bp, Artificial
  Synthetic
ccatctcatccctgc

SEQ ID NO: 185 - DNA, 16 bp, Artificial
  Synthetic
ccatctcatccctgcg

SEQ ID NO: 186 - DNA, 22 bp, Artificial
  Synthetic
tttttttaatttgggtttagtt

SEQ ID NO: 187 - DNA, 20 bp, Artificial
  Synthetic
acctaccccactaccaaaac

SEQ ID NO: 188 - DNA, 165 bp, Homo sapiens
tccctctaacctgggtccagcccggctggagatgggtgggagtgcgacctagggctggcgggcaggcgggcactgtgtctccctgactgtgtcctcctgtgtccctctgcctcgccgctgttccggaacctgctctgcgcggcacgtcctggcagtggggcaggt

SEQ ID NO: 189 - DNA, 1431 bp, Homo sapiens
agccctccaggacaggctgcatcagaagaggccatcaagcaggtctgttccaagggcctttgcgtcaggtgggctcaggattccagggtggctggaccccaggccccagctctgcagcagggaggacgtggctgggctcgtgaagcatgtgggggtgagcccaggggccccaaggcagggcacctggccttcagcctgcctcagccctgcctgtctcccagatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcagggtgagccaactgcccattgctgcccctggccgcccccagccaccccctgctcctggcgctcccacccagcatgggcagaagggggcaggaggctgccacccagcagggggtcaggtgcacttttttaaaaagaagttctcttggtcacgtcctaaaagtgaccagctccctgtggcccagtcagaatctcagcctgaggacggtgttggcttcggcagccccgagatacatcagagggtgggcacgctcctccctccactcgcccctcaaacaaatgccccgcagcccatttctccaccctcatttgatgaccgcagattcaagtgttttgttaagtaaagtcctgggtgacctggggtcacagggtgccccacgctgcctgcctctgggcgaacaccccatcacgcccggaggagggcgtggctgcctgcctgagtgggccagacccctgtcgccaggcctcacggcagctccatagtcaggagatggggaagatgctggggacaggccctggggagaagtactgggatcacctgttcaggctcccactgtgacgctgccccggggcgggggaaggaggtgggacatgtgggcgttggggcctgtaggtccacacccagtgtgggtgaccctccctctaacctgggtccagcccggctggagatgggtgggagtgcgacctagggctggcgggcaggcgggcactgtgtctccctgactgtgtcctcctgtgtccctctgcctcgccgctgttccggaacctgctctgcgcggcacgtcctggcagtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcccgcaggcagccccacacccgccgcctcctgcaccgagagagatggaataaagcccttgaaccagc

(19) United States
(12) Patent Application Publication
(10) Pub. No.: US 2022/0064731 A1
(43) Pub. Date: Mar. 3, 2022

(54) PRIMERS FOR MULTIPLEX PCR

(71) Applicants: YISSUM RESEARCH DEVELOPMENT COMPANY OF THE HEBREW UNIVERSITY OF JERUSALEM LTD., Jerusalem (IL); HADASIT MEDICAL RESEARCH SERVICES & DEVELOPMENT LTD., Jerusalem (IL)

(72) Inventors: Yuval DOR, Jerusalem (IL); Ruth SHEMER, Mevasseret Zion (IL); Daniel NEIMAN, Bnei Dekalim (IL); Benjamin GLASER, Jerusalem (IL)

(21) Appl. No.: 17/601,069

(22) PCT Filed: Apr. 2, 2020
(86) PCT No.: PCT/IL2020/050405

§ 371 Date: Oct. 3, 2021

Related U.S. Application Data
(60) Provisional application No. 62/828,587, filed on Apr. 3, 2019.

(51) Int. Cl. C12Q 1/6881; C12Q 1/686

(52) U.S. Cl. CPC C12Q 1/6881; C12Q 2600/154; C12Q 2600/16; C12Q 1/686

(57) ABSTRACT

Primer sets comprising a 3′ region of homology to a 5′ segment of a target nucleic acid molecule and a 5′ first adapter sequence wherein the first adapter sequence comprises a 5′ first and 3′ second region and wherein said first region is between 50% and 99% of the first adapter sequence; and a second primer comprising a 3′ region identical to the first region of the first adapter sequence and a 5′ second adapter sequence are provided. Methods of performing polymerase chain reaction (PCR), producing a sequencing library and determining the cell type of origin of cell free DNA (cfDNA) are also provided.

### CROSS REFERENCE TO RELATED APPLICATIONS

This application claims the benefit of priority of U.S. Provisional Patent Application No. 62/828,587, filed Apr. 3, 2019, the contents of which are all incorporated herein by reference in their entirety.

### FIELD OF INVENTION

The present invention is in the field of multiplex PCR.

### BACKGROUND OF THE INVENTION

PCR amplification followed by sequencing is a powerful diagnostic tool that enables identification of point mutations, disease states, tissue origins and wide variety of other information. However, when small initial samples are present it can be difficult to successfully probe multiple genomic loci. Multiplex PCR facilitates the examination of many loci simultaneously but can be difficult to couple to sequencing technologies. Methods and molecules that allow for robust, reliable and repeatable multiplex PCR coupled to next generation sequencing are thus greatly needed.

### SUMMARY OF THE INVENTION

The present invention provides primer sets comprising a first primer with a segment specific to a target and a second primer with an overlapping region to the first primer. The present invention further concerns methods of performing multiplex polymerase chain reaction (PCR), and methods of producing a sequencing library with the primers of the invention. Methods of determining the cell type of origin of cell free DNA (cfDNA) are also provided.

According to a first aspect, there is provided a primer set comprising:
a. a first primer comprising a 3′ region of homology to a 5′ segment of a target nucleic acid molecule and a 5′ first adapter sequence wherein the first adapter sequence comprises a 5′ first and 3′ second region and wherein the first region is between 50% and 99% of the first adapter sequence; and
b. a second primer comprising a 3′ region identical to the first region of the first adapter sequence and a 5′ second adapter sequence.

According to another aspect, there is provided a kit, comprising at least two primer sets of the invention, wherein each first primer comprises a 3′ region of homology to a different target nucleic acid molecule and
a. wherein the second adapter sequence is a universal sequence shared by all second primers;
b. wherein the fourth adapter sequence is a universal sequence shared by all fourth primers, or
c. both a and b.

According to another aspect, there is provided a method of polymerase chain reaction (PCR), the method comprising:
a. providing a sample comprising a target nucleic acid molecule;
b. performing a first PCR reaction with the target nucleic acid molecule and a first primer of a primer set of the invention to produce a first adapter labeled target nucleic acid hybrid molecule; and
c. performing a second PCR reaction with the hybrid molecule and a second primer of a primer set of the invention to produce a first and second adapter labeled target nucleic acid hybrid molecule.

According to another aspect, there is provided a method of generating a sequencing library, the method comprising:
a. providing a sample comprising at least two target nucleic acid molecules;
b. performing a first PCR reaction with the at least two target nucleic acid molecules and at least two first primers of a primer set of the invention to produce first adapter labeled target nucleic acid hybrid molecules, wherein the at least two first primers are homologous to different target nucleic acid molecules and comprise identical first regions of a first adapter; and
c. performing a second PCR reaction with the hybrid molecules and a second primer of the primer set of the invention to produce first and second adapter labeled target nucleic acid hybrid molecules;

thereby generating a sequencing library.

According to another aspect, there is provided a method of determining the cell type of origin of cfDNA, the method comprising:
a. providing a sample comprising at least two target nucleic acid molecules, wherein the at least two target nucleic acid molecules are cfDNA, that comprise at least one cell type-specific methylation/unmethylated site and wherein the at least two target nucleic acid molecules of cfDNA have undergone bisulfate conversion;
b. performing a first PCR reaction with the at least two target nucleic acid molecules and at least two first primers to produce first adapter labeled target nucleic acid hybrid molecules, wherein each of the first primers comprises
   i. a 3′ region of homology to a 5′ segment of one of the at least two target nucleic acid molecules; and
   ii. a 5′ first adapter sequence common to all first primers wherein the first adapter sequence comprises a 5′ first and 3′ second region;
c. performing a second PCR reaction with the first primer labeled hybrid molecules and at least two second primers to produce first and second adapter labeled target nucleic acid hybrid molecules, wherein each of the second primers comprises
   i. a 3′ region identical to the first region of the first adapter sequence; and
   ii. a 5′ second adapter sequence;
d. sequencing the first and second adapter labeled target nucleic acid molecules; and
e. determining a methylation status of the methylation/unmethylated site according to the base sequenced at the methylation/unmethylated site;
wherein the presence of a methylation mark or lack of a methylation mark that is cell type-specific indicates that the cfDNA originates from the cell type, thereby determining the cell type of origin of the cfDNA.

According to some embodiments, the primer set of the invention further comprises a third primer comprising a 3′ region that is a reverse compliment to a 3′ segment of the target nucleic acid molecule and suitable for amplifying the target molecule in combination with the first primer.

According to some embodiments, the primer set of the invention further comprises a fourth primer, wherein the third primer further comprises a 5′ third adapter sequence wherein the third adapter sequence comprises a first and second region and wherein the fourth primer comprises a 3′ region identical to the first region of the third adapter sequence and a 5′ fourth adapter sequence.

According to some embodiments, the first region of the third adapter sequence is between 50% and 99% of the third adapter sequence.

According to some embodiments, the second region of the first adapter sequence and the second region of the third adapter sequence are at least 85% identical.

According to some embodiments, the 3′ region of the second primer and/or the fourth primer is less than 35% of the second and/or fourth primer.

According to some embodiments, the first region of the first adapter and/or the first region of the third adapter is between 14 and 19 nucleotides.

According to some embodiments, the second region of the first adapter and/or the second region of the third adapter is between 7 to 11 nucleotides.

According to some embodiments, the second primer, the fourth primer or both further comprises a barcode 5′ of the 3′ region.

According to some embodiments, the first adapter sequence comprises the sequence TCCCTACACGACGCTCTTCCGATCT (SEQ ID NO: 1).

According to some embodiments, the third adapter sequence comprises the sequence AGTTCAGACGTGTGCTCTTCCGATC (SEQ ID NO: 2).

According to some embodiments, the second region of the first adapter and/or the second region of the third adapter comprises the sequence TTCCGATC (SEQ ID NO: 3).

According to some embodiments, the sample comprises cell free DNA (cfDNA).

According to some embodiments, the sample comprises bisulfate converted nucleic acids.

According to some embodiments, the at least two target nucleic acid molecules are a first and second strand of a target double stranded bisulfate converted DNA.

According to some embodiments, the method of the invention further comprises sequencing the first and second adapter labeled nucleic acids of the sequencing library.

According to some embodiments, the first PCR is performed with at least 3 first primers that each comprise a region of homology to a target nucleic acid molecule with a cell type-specific methylation/unmethylated site for a different cell type.

According to some embodiments, the first PCR is performed with at least 3 first primers that each comprise a region of homology to a target nucleic acid molecule with a different cell type-specific methylation/unmethylated site for the same cell type.

According to some embodiments, the first PCR is performed with a first first primer with homology to a forward strand of a nucleic acid molecule with a cell type-specific methylation/unmethylated site and a second first primer with homology to a reverse strand of the same nucleic acid molecule with a cell-type specific methylation/unmethylated site.

According to some embodiments, the method is performed with a primer set of the invention.

According to some embodiments, the first PCR reaction comprises 15 to 25 cycles and wherein the PCR reaction is a gradient reaction wherein the annealing temperature increases during the first PCR reaction.

According to another aspect, there is provided a method of detecting cell free DNA (cfDNA) in a sample, the method comprising:
a. receiving a sample comprising cfDNA; and
b. detecting in the sample a DNA sequence of a region of an insulin (INS) gene, wherein the region is between nucleotides 1058-1222 downstream of an INS transcriptional start site and comprises at least one cytosine base selected from cytosine 1080, 1102, 1116, 1124, 1170, 1173, 1181, 1195, 1197 and 1202;

thereby detecting cfDNA in a sample.

According to another aspect, there is provided a method of detecting beta cell cfDNA in a sample, the method comprising:
a. receiving a sample comprising cfDNA;
b. detecting in the sample a DNA sequence of a region of an insulin (INS) gene, wherein the region is between nucleotides 1058-1222 downstream of an INS transcriptional start site and comprises at least one cytosine base selected from cytosine 1080, 1102, 1116, 1124, 1170, 1173, 1181, 1195, 1197 and 1202; and
c. determining a methylation status of the at least one cytosine base, wherein absence of methylation on the at least one cytosine base indicates the presence of beta cell cfDNA;
thereby detecting beta cell cfDNA in a sample.

According to some embodiments, the sample is a blood sample or isolated cfDNA from a blood sample.

According to some embodiments, the cfDNA is unmethylated cfDNA.

According to some embodiments, the sample comprises methylation sensitive converted cfDNA, wherein unmethylated cytosine residues of the cfDNA are converted to thymine and methylated cytosine residues of the cfDNA are unconverted.

According to some embodiments, the methylation sensitive converted cfDNA has undergone bisulfite conversion.

According to some embodiments, the detection comprises sequencing of the region, PCR amplification of the region, methylation-specific PCR amplification of the region, or a combination thereof.

According to some embodiments, the region comprises a plurality of cytosine bases selected from cytosine 1080, 1102, 1116, 1124, 1170, 1173, 1181, 1195, 1197 and 1202.

According to some embodiments, the INS gene consists of the sequence of SEQ ID NO: 189.

According to some embodiments, the method further comprises determining the methylation status of said at least one cytosine base.

According to some embodiments, the sample is from a subject and the detecting beta cell cfDNA comprises detecting beta cell death in the subject.

According to some embodiments, the beta cell death is detection of a beta cell-associated pathology in the subject.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-C: (1A) A schematic representation of the PCR of the invention. (1B) A schematic representation and sequences of one possible embodiment of primers of the invention. (1C) A schematic representation of a PCR of the invention on both strands of a double-stranded bisulfite converted molecule.

FIG. 2A-E: (2A) A bar chart showing he specificity of beta cell specific methylation markers to beta cell DNA. (2B-C) Bar charts of relative expression of 5 beta cell specific markers from sample with (2B) various number of beta cell genomes spiked in and (2C) various percentages of the sample being beta cell genomes. (2D) A bar chart of relative expression of sense and antisense strands from a beta cell specific locus of the insulin gene. (2E) A dot plot of relative expression of the two strands from FIG. 2D.

FIG. 3: A bar chart of the relative expression of ten loci (5 beta cell-specific, 2 exocrine pancreas-specific, 3 colon-specific) in bisulfite converted cfDNA from seven healthy controls and 5 islet transplant recipients.

FIG. 4A-C: (4A) A bar chart of relative expression of nine pancreas/beta cell specific markers from samples with various amounts of starting bisulfite converted cfDNA from an islet transplant recipient. The left column shows expression when each locus was amplified individually, and the middle and right columns show expression from multiplex PCR. (4B-C) Bar charts of relative expression of (4B) of ten unmethylated loci found in healthy cfDNA and (4C) 30 loci examined in multiplex reactions with varying numbers of primer pairs.

FIGS. 5A-C: Specificity and sensitivity of beta cell methylation markers. (5A) Tissue specificity of 5 methylation markers of human beta cells identified using comparative methylome analysis. Note that markers near the insulin and Leng8 genes are unmethylated in a proportion of pancreatic acinar cells, and that insulin is unmethylated in ˜10% of DNA molecules in the intestine. (5B) Sensitivity of a 6-marker beta cell panel in identifying beta cell DNA embedded in blood DNA, based on fraction of beta cell genomes. Six beta cell markers (the 5 described in A and the insulin antisense, having the same specificity as insulin but representing independent molecules) were amplified and sequenced in mixtures of blood DNA and DNA from sorted primary beta cells in the indicated proportions. All samples included 60 pg beta cell DNA (10 genome equivalents), mixed with 6 ng to 180 ng of leukocyte DNA. (5C) Sensitivity of the 6-marker beta cell panel based on absolute number of beta cell molecules. The indicated numbers of beta cell genomes were mixed into 10 ng of blood DNA.

FIGS. 6A-C: Analysis of insulin gene methylation. (6A) Schematic of CpG sites in the insulin gene, relative to the transcription start site, along with indication of publications that used each site or sites for cfDNA analysis. (6B) Bar chart of the percentage of unmethylated molecules from each region of the insulin gene, scoring molecules that are unmethylated in all cytosines, in sorted human beta cells and acinar cells. The downstream area of the gene (INS10) appears to have a beta-cell-specific demethylation. (6C) Bar chart of unmethylated insulin in intestinal tissue, colorectal cancer and plasma of patients with colorectal cancer. Graph shows the percentage of unmethylated insulin molecules that are present in DNA from a normal intestine (samples from 2 individuals), colorectal cancer (CRC, n=6), and cfDNA from patients with advanced CRC (n=5).

FIGS. 7A-B: Baseline levels of beta cell-derived cfDNA in healthy individuals of different ages. (7A) Accumulated levels of 6 beta cell markers in plasma samples from individuals at the indicated age. Local young healthy controls, n=36; local adult healthy controls, n=85. Dotted red line, average (of cumulative values of all 6 markers)+2SD of all healthy controls. (7B) Beta cell cfDNA levels in healthy individuals, calculated as average of the 6 markers per individual, presented as a function of donor age in years.

FIGS. 8A-B: Levels of beta cell-derived cfDNA in islet transplant recipients. Islet transplant recipients were sampled ˜1 hour after transplantation. (8A) Levels of individual markers, demonstrating identification of all 6 markers in most samples. (8B) Average levels of beta cell-derived cfDNA in islet transplant recipients vs. healthy controls (n=85) (P<0.0001, Mann-Whitney test).

FIG. 9: Measurement of beta cell-derived cfDNA in a child with congenital hyperinsulinism. Patient was sampled at 5 different times (age 9 months-2.5 years), and compared with 22 age-matched controls. DoAtted red line, average+2SD of signal from healthy controls.

FIGS. 10A-D: Using multiple markers in multiplex PCR increases the assay specificity. Bar charts showing the detection of seven markers specific for (10A) cardiomyocytes, (10B) colon cells, (10C) pancreatic duct cells and (10D) breast cells. Each color represents one marker.

FIGS. 11A-D: Using multiple markers in multiplex PCR increases the assay sensitivity. Bar charts showing the detection of (11A-B) seven markers specific for cardiomyocytes or (11C-D) twelve markers specific for brain cells in blood cfDNA spiked with cardiomyocyte DNA or brain DNA respectively. The amount of spiked in DNA is shown as (11A, 11C) the percentage of genome equivalents present and (11B, 11D) the total number of cells. Each color represents one marker.

### DETAILED DESCRIPTION OF THE INVENTION

The present invention, in some embodiments, provides primer sets comprising a first primer with a segment specific to a target and a second primer with an overlapping region to the first primer. The present invention further concerns methods of performing polymerase chain reaction (PCR), and methods of producing a sequencing library with the primers of the invention. Methods of determining the cell type of origin of cell free DNA (cfDNA) are also provided.

The invention is based on the unexpected finding that a limited region of overlap between the 5′ end of primers from a first step PCR reaction and the 3′ end of the primers from the second step PCR reaction can greatly enhance efficacy of a multiple PCR reaction. The inventors found that with a small starting amount of template, as many as 30 separate PCR reactions can be run on the same sample. This is done with primer pairs for the 30 reactions all having the same adapter sequences on the forward and reverse primers. The second, universal, PCR reaction uses primers that only partially overlap with these adapter sequences. Specifically, the inventors found that an overlap of 13-20 nucleotides, or around 50-99% of the adapter region, produced more robust and accurate results. Smaller overlaps could not insure fidelity of the reaction and larger overlaps decreased PCR efficiency. This method of multiple PCR was found to be advantageous for methylome analysis (where information from both strands in advantageous), for cfDNA analysis (wherein very small amounts of template are available), and especially for methylome analysis of cfDNA. By using the TruSeq Universal Adapter sequence and breaking it into two primers with the proper overlap, the products from the multiplex PCR are produced ready for sequencing without the need for a ligation step.

By a first aspect, there is provided a primer set comprising:
a. a first primer comprising:
   i. a 3′ region of homology to a 5′ segment of a target nucleic acid molecule and
   ii. a 5′ first adapter sequence, wherein the first adapter sequence comprising a first and second region;
b. a second primer comprising:
   i. a 3′ region identical to the first region of the first adapter sequence, and
   ii. a 5′ second adapter sequence.

As used herein, the term “primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. Primers within the scope of the present invention bind adjacent to a target sequence. A “primer” may be considered a short polynucleotide, generally with a free 3′-OH group that binds to a target or template potentially present in a sample of interest by hybridizing with the target, and thereafter promoting polymerization of a polynucleotide complementary to the target.

In some embodiments, homology is perfect homology. In some embodiments, homology comprises complementarity. In some embodiments, homology is reverse complementarity. In some embodiments, the region of homology comprises at most 1, 2, 3, 4, 5, 6, or 7 mismatches. Each possibility represents a separate embodiment of the invention. In some embodiments, the region of homology can be itself a primer for amplification of the target nucleic acid. In some embodiments, the region of homology comprises or consists of at least 10, 12, 15, 17, 20, 22, 25, 27 or 30 nucleotides. Each possibility represents a separate embodiment of the invention. In some embodiments, the region of homology comprises or consists of between 10-35, 10-30, 10-27, 10-25, 10-22, 10-20, 12-35, 12-30, 12-27, 12-25, 12-22, 12-20, 15-35, 15-30, 15-27, 15-25, 15-22, 15-20, 17-35, 17-30, 17-27, 17-25, 17-22, or 17-20, nucleotides. Each possibility represents a separate embodiment of the invention. In some embodiments, the region of homology comprises or consists of between 20-27 nucleotides.

Design of primers for amplification of specific genes is well known in the art, and such primers can be found or designed on various websites such as bioinfo.ut.ee/primer3-0.4.0/or pga.mgh.harvard.edu/primerbank/ for example. In some embodiments, the region of homology complies with rules governing the composition of a primer. In some embodiments, the region of homology complies with rules governing primer design for after bisulfite sequencing. After bisulfite sequencing unmethylated cytosines have been converted to thymine. As such, primers are designed for regions that do not contain cytosines (since methylation status is being examined it is unknown if the sequence would have a cytosine or a thymine), and specifically the primers are limited to only having three bases: thymine, guanine and adenine. As guanine and cytosine content is a driving factor for the melting temperature (Tm) of the primer, bisulfite primers tend to have lower Tms (due to the absence of cytosines). Tm is increased proportionally to primer length; thus, the adapter overhangs will increase the Tm in future rounds of PCR. This makes the primers of the invention particularly suited for multiplex PCR after bisulfite conversion. Further, gradient PCR in which the annealing temperature increases is also particularly suited for the initial PCR reaction.

In some embodiments, the target nucleic acid molecule is a sequence to be amplified. In some embodiments, the amplification is polymerase chain reaction (PCR). In some embodiments, the PCR is real-time PCR. In some embodiments, the PCR is quantitative (qPCR). In some embodiments, the PCR is multiplex PCR. In some embodiments, the target nucleic acid is cDNA. In some embodiments, the target nucleic acid is cell-free DNA (cfDNA). In some embodiments, the target nucleic acid is RNA. In some embodiments, the target nucleic acid is a bisulfite converted nucleic acid molecule.

The terms “bisulfite conversion” and “sodium bisulfite conversion” are used herein synonymously and refer to a technique for converting unmethylated cytosine into uracil or thymine and leaving methyl-cytosine intact. Thus, when sequencing is performed methyl-cytosine is read as a “C”, whereas unmethylated cytosine is read as a “T” or “U”. This allows for precise mapping of DNA methylation. In some embodiments, the methyl-cytosine is 5-methylcytosine. Bisulfite conversion kits can be purchased commercially, such as for example, the EZ DNA methylation kits from Zymo Research, the EpiMark Bisulfite conversion kit from NEB, the EpiTect Bisulfite kits from Qiagen and the EpiJET bisulfite conversion kit from Thermo Fisher.

In some embodiments, the region of homology is the 3′ end of the first primer. In some embodiments, the first adapter sequence is the 5′ end of the first primer. In some embodiments, the first primer comprises the region of homology and the first adapter sequence. In some embodiments, the first primer consists of the region of homology and the first adapter sequence.

In some embodiments, the region of homology to a target sequence is added 3′ to the first adapter. In some embodiments, the region of homology to a target sequence is directly adjacent to the first adapter. In some embodiments, there is a linker between the region of homology to a target sequence and the first adapter.

In some embodiments, a linker is a nucleotide linker. In some embodiments, a nucleotide linker is 1, 2, 3, 4, 5, 6, 7, 8, 9, or 10 nucleotides. Each possibility represents a separate embodiment of the invention. In some embodiments, a nucleotide linker is at least 1, 2, 3, 4, 5, 6, 7, 8, 9, or 10 nucleotides. Each possibility represents a separate embodiment of the invention. In some embodiments, a nucleotide linker is at most 1, 2, 3, 4, 5, 6, 7, 8, 9, 10, 11, 12, 13, 14, 15, 16, 17, 18, 19 or 20 nucleotides. Each possibility represents a separate embodiment of the invention. In some embodiments, a nucleotide linker is at most 5, 10, 15, 20, 25, 30, 35, 40, 45, 50, 55, 60, 65, 70, 75, 80, 85, 90, 95, 97, 99 or 100% of the length of the first adapter sequence. Each possibility represents a separate embodiment of the invention.

In some embodiments, the first adapter sequence comprises or consists of at least 10, 12, 15, 17, 20, 22, 25, 27 or 30 nucleotides. Each possibility represents a separate embodiment of the invention. In some embodiments, the first adapter sequence comprises or consists of between 10-35, 10-30, 10-27, 10-25, 10-22, 10-20, 12-35, 12-30, 12-27, 12-25, 12-22, 12-20, 15-35, 15-30, 15-27, 15-25, 15-22, 15-20, 17-35, 17-30, 17-27, 17-25, 17-22, 17-20, 20-35, 20-30, 20-27, 20-25, 20-22, 22-35, 22-30, 22-27, 22-25, 24-35, 24-30, 24-27, 24-26, 24-25, 25-35, 25-30, 25-27, or 25-26 nucleotides in length. Each possibility represents a separate embodiment of the invention. In some embodiments, the first adapter sequence comprises or consists of between 24-26 nucleotides. In some embodiments, the first adapter sequence consists of 25 nucleotides.

In some embodiments, the first region of the first adapter sequence is 5′ to the second region of the first adapter sequence. In some embodiments, the first region is the 5′ end of the first adapter sequence. In some embodiments, the second region is the 3′ end of the first adapter sequence. In some embodiments, the first region of the first adapter is larger than the second region of the first adapter. In some embodiments, the first region of the first adapter is larger than or equal to the second region of the first adapter. In some embodiments, the first region is 10-100, 20-100, 30-100, 40-100, 50-100, 60-100, 65-100, 10-99, 20-99, 30-99, 40-99, 50-99, 60-99, 65-99, 10-95, 20-95, 30-95, 40-95, 50-95, 60-95, 65-95, 10-90, 20-90, 30-90, 40-90, 50-90, 60-90, 65-90, 10-85, 20-85, 30-85, 40-85, 50-85, 60-85, 65-85, 10-80, 20-80, 30-80, 40-80, 50-80, 60-80, 65-80, 10-75, 20-75, 30-75, 40-75, 50-75, 60-75, 65-75, 10-70, 20-70, 30-70, 40-70, 50-70, 60-70, or 65-70% of the first adapter sequence. Each possibility represents a separate embodiment of the invention. In some embodiments, the first region is 50-99% of the first adapter sequence. In some embodiments, the first region is 50-100% of the first adapter sequence.

In some embodiments, the first region of the first adapter comprises at least 3, 5, 7, 9, 10, 12, 14, 15, or 17 nucleotides. Each possibility represents a separate embodiment of the invention. In some embodiments, the first region of the first adapter comprises at most 17, 19, 20, 21, 23, 25, 27, 29, 30, 32, 34 or 35 nucleotides. Each possibility represents a separate embodiment of the invention. In some embodiments, the first region of the first adapter comprises between 5-30, 5-27, 5-25, 5-23, 5-20, 5-19, 5-18, 5-17, 7-30, 7-27, 7-25, 7-23, 7-20, 7-19, 7-18, 7-17, 10-30, 10-27, 10-25, 10-23, 10-20, 10-19, 10-18, 10-17, 12-30, 12-27, 12-25, 12-23, 12-20, 12-19, 12-18, 12-17, 14-30, 14-27, 14-25, 14-23, 14-20, 14-19, 14-18, 14-17, 15-30, 15-27, 15-25, 15-23, 15-20, 15-19, 15-18, 15-17, 16-30, 16-27, 16-25, 16-23, 16-20, 16-19, 16-18, or 16-17 nucleotides. Each possibility represents a separate embodiment of the invention. In some embodiments, the first region of the first adapter comprises 15-18 nucleotides. In some embodiments, the first region of the first adapter comprises 16 or 17 nucleotides.

In some embodiments, the second region of the first adapter sequence is 3′ to the first region of the first adapter sequence. In some embodiments, the second region of the first adapter is smaller than the first region of the first adapter. In some embodiments, the second region of the first adapter is smaller than or equal to the first region of the first adapter. In some embodiments, the second region is 1-50, 1-40, 1-30, 1-20, 1-10, 1-5, 5-50, 5-40, 5-30, 5-20, 5-10, 10-50, 10-40, 10-30, 10-20, 15-50, 15-40, 15-30, 15-20, 20-50, 20-40, 20-30, 25-50, 25-40 or 25-30% of the first adapter sequence. Each possibility represents a separate embodiment of the invention. In some embodiments, the second region is 1-50% of the first adapter sequence.

In some embodiments, the second region of the first adapter comprises at least 1, 3, 5, 7, 8, 9, 10, 11, 12, 14, 15, or 17 nucleotides. Each possibility represents a separate embodiment of the invention. In some embodiments, the second region of the first adapter comprises at most 8, 9, 10, 11, 12, 15, 17, 19, 20, 21, 23 or 25 nucleotides. Each possibility represents a separate embodiment of the invention. In some embodiments, the second region of the first adapter comprises between 1-15, 1-13, 1-10, 1-9, 1-8, 2-15, 2-13, 2-10, 2-9, 2-8, 3-15, 3-13, 3-11, 3-10, 3-9, 3-8, 5-15, 5-13, 5-10, 5-9, 5-8, 6-15, 6-13, 6-10, 6-9, 6-8, 7-15, 7-13, 7-10, 7-9, 7-8, 8-15, 8-13, 8-10 or 8-9 nucleotides. Each possibility represents a separate embodiment of the invention. In some embodiments, the second region of the first adapter comprises 6-10 nucleotides. In some embodiments, the second region of the first adapter comprises 8 nucleotides.

In some embodiments, the first adapter comprises and/or consists of the sequence TCCCTACACGACGCTCTTCCGATCT (SEQ ID NO: 1). In some embodiments, the first adapter comprises and/or consists of the sequence AGTTCAGACGTGTGCTCTTCCGATC (SEQ ID NO: 2). In some embodiments, the first region of the first adapter comprises and/or consists of the sequence TCCCTACACGACGCTC (SEQ ID NO: 3). In some embodiments, the second region of the first adapter comprises and/or consists of the sequence TTCCGATCT (SEQ ID NO: 4). In some embodiments, the first region of the first adapter comprises and/or consists of the sequence AGTTCAGACGTGTGCTC (SEQ ID NO: 5). In some embodiments, the second region of the first adapter comprises and/or consists of the sequence TTCCGATC (SEQ ID NO: 6).

In some embodiments, the first adapter comprises and/or consists of the sequence ATGGGCAGTCGGTGAT (SEQ ID NO: 138). In some embodiments, the first region of the first adapter comprises and/or consists of the sequence ATGGGCAGTCGGTGA (SEQ ID NO: 139). In some embodiments, the second region of the first adapter comprises and/or consists of the nucleotide T. In some embodiments, the first region of the first adapter consists of the entire first adapter. In some embodiments, the first adapter comprises and/or consists of the sequence TCTATGGGCAGTCGGTGAT (SEQ ID NO: 140). In some embodiments, the first region of the first adapter comprises and/or consists of the sequence TCTATGGGCAGTCGG (SEQ ID NO: 141). In some embodiments, the first region of the first adapter comprises and/or consists of the sequence TCTATGGGCAGTCGGT (SEQ ID NO: 142). In some embodiments, the first region of the first adapter comprises and/or consists of the sequence TCTATGGGCAGTCGGTG (SEQ ID NO: 143). In some embodiments, the first region of the first adapter comprises and/or consists of the sequence TCTATGGGCAGTCGGTGA (SEQ ID NO: 144). In some embodiments, the second region of the first adapter comprises and/or consists of the sequence TGAT, GAT, AT or T. Each possibility represents a separate embodiment of the invention. In some embodiments, the first adapter comprises and/or consists of the sequence GGGCAGTCGGTGAT (SEQ ID NO: 145). In some embodiments, the first adapter comprises and/or consists of the sequence TGTCTCCGACTCAG (SEQ ID NO: 146). In some embodiments, the first region of the first adapter comprises and/or consists of the sequence TGTCTCCGACTCA (SEQ ID NO: 147). In some embodiments, the second region of the first adapter comprises and/or consists of the nucleotide G. In some embodiments, the first region is the entire first adapter. In some embodiments, the first adapter comprises and/or consists of the sequence TGCGTGTCTCCGACTCAG (SEQ ID NO: 148). In some embodiments, the first region of the first adapter comprises and/or consists of the sequence TGCGTGTCTCCGACT (SEQ ID NO: 149). In some embodiments, the first region of the first adapter comprises and/or consists of the sequence TGCGTGTCTCCGACTC (SEQ ID NO: 150). In some embodiments, the first region of the first adapter comprises and/or consists of the sequence TGCGTGTCTCCGACTCA (SEQ ID NO: 151). In some embodiments, the first adapter comprises and/or consists of the sequence GCGTGTCTCCGACTCAG (SEQ ID NO: 152). In some embodiments, the first region of the first adapter comprises and/or consists of the sequence GCGTGTCTCCGACT (SEQ ID NO: 153). In some embodiments, the first region of the first adapter comprises and/or consists of the sequence GCGTGTCTCCGACTC (SEQ ID NO: 154). In some embodiments, the first region of the first adapter comprises and/or consists of the sequence GCGTGTCTCCGACTCA (SEQ ID NO: 155). In some embodiments, the first adapter comprises and/or consists of the sequence CGTGTCTCCGACTCAG (SEQ ID NO: 156). In some embodiments, the first region of the first adapter comprises and/or consists of the sequence CGTGTCTCCGACT (SEQ ID NO: 157). In some embodiments, the first region of the first adapter comprises and/or consists of the sequence CGTGTCTCCGACTC (SEQ ID NO: 158). In some embodiments, the first region of the first adapter comprises and/or consists of the sequence CGTGTCTCCGACTCA (SEQ ID NO: 159). In some embodiments, the first adapter comprises and/or consists of the sequence GTGTCTCCGACTCAG (SEQ ID NO: 160). In some embodiments, the first region of the first adapter comprises and/or consists of the sequence GTGTCTCCGACT (SEQ ID NO: 161). In some embodiments, the first region of the first adapter comprises and/or consists of the sequence GTGTCTCCGACTC (SEQ ID NO: 162). In some embodiments, the first region of the first adapter comprises and/or consists of the sequence GTGTCTCCGACTCA (SEQ ID NO: 163). In some embodiments, the second region of the first adapter comprises and/or consists of the sequence CAG, AG or G. Each possibility represents a separate embodiment of the invention.

In some embodiments, the second primer comprises a region similar to the first region of the first adapter. In some embodiment similar is identical. In some embodiments, similar is homologous. In some embodiments, similar is complementary. In some embodiments, a similar region comprises at most 1, 2, 3, 4, 5, 6, or 7 mismatches. In some embodiments, a similar region is identical to the first region of the first adapter. In some embodiments, the region similar to first region of the first adapter is 3′ to the second adapter sequence. In some embodiments, the region similar to first region of the first adapter is at the 3′ end of the second primer. In some embodiments, a region similar to the first region of the first adapter comprises and/or consists of SEQ ID NO: 3 or SEQ ID NO: 5. In some embodiments, a region similar to the first region of the first adapter is selected from SEQ ID NO: 3 and SEQ ID NO: 5. In some embodiments, a region similar to the first region of the first adapter comprises and/or consists of SEQ ID NO: 3, SEQ ID NO: 5, SEQ ID NO: 141, SEQ ID NO: 142, SEQ ID NO: 143, SEQ ID NO: 144, SEQ ID NO: 147, SEQ ID NO: 148, SEQ ID NO: 149, SEQ ID NO: 150, SEQ ID NO: 151, SEQ ID NO: 153, SEQ ID NO: 154, SEQ ID NO: 155, SEQ ID NO: 157, SEQ ID NO: 158, SEQ ID NO: 159, SEQ ID NO: 161, SEQ ID NO: 162 or SEQ ID NO: 163. Each possibility represents a separate embodiment of the invention. In some embodiments, a region similar to the first region of the first adapter is selected from SEQ ID NO: 3, SEQ ID NO: 5, SEQ ID NO: 141, SEQ ID NO: 142, SEQ ID NO: 143, SEQ ID NO: 144, SEQ ID NO: 147, SEQ ID NO: 148, SEQ ID NO: 149, SEQ ID NO: 150, SEQ ID NO: 151, SEQ ID NO: 153, SEQ ID NO: 154, SEQ ID NO: 155, SEQ ID NO: 157, SEQ ID NO: 158, SEQ ID NO: 159, SEQ ID NO: 161, SEQ ID NO: 162 and SEQ ID NO: 163.

In some embodiments, the second adapter sequence is 5′ to the similar region. In some embodiments, the second adapter sequence is the 5′ end of the second primer. In some embodiments, the second adapter sequence is a universal sequence. In some embodiments, the second adapter sequence comprises a primer sequence for a sequencing primer. In some embodiments, the primer sequence is complementary to a sequencing primer. In some embodiments, the second adapter sequence comprises at least 10, 15, 20, 25, 30, or 35 nucleotides. Each possibility represents a separate embodiment of the invention. In some embodiments, the second adapter sequence comprises at most 30, 35, 40, 45, 50, 55, or 60 nucleotides. Each possibility represents a separate embodiment of the invention. In some embodiments, the second adapter sequence comprises between 20-50, 25-50, 30-50, 31-50, 32-50, 33-50, 35-50, 20-45, 25-45, 30-45, 31-45, 32-45, 33-45, 35-45, 20-40, 25-40, 30-40, 31-40, 32-40, 33-40, 35-40, 20-35, 25-35, 30-35, 31-35, 32-35, 33-35, 20-34, 25-34, 30-34, 31-34, 32-34, or 33-34 nucleotides. Each possibility represents a separate embodiment of the invention.

In some embodiments, the second primer comprises and/or consists of the sequence AATGATACGGCGACCACCGAGATCTACACTCTTTCCCTACACGACGCTC (SEQ ID NO: 7). In some embodiments, the second primer comprises and/or consists of the sequence CAAGCAGAAGACGGCATACGAGATGTGACTGGAGTTCAGACGTGTGCTC (SEQ ID NO: 9). In some embodiments, the second primer comprises an indexing sequence. In some embodiments, the indexing sequence is a barcode sequence. In some embodiments, the second primer comprises and/or consists of the sequence CAAGCAGAAGACGGCATACGAGAGTGACTGGAGTTCAGACGTGTG CTC (SEQ ID NO: 8). In some embodiments, the barcode/index sequence is a unique six nucleotide sequence that identifies PCR products produced by the second primer.

In some embodiments, the second primer comprises the sequence CCACTACGCCTCCGCTTTCCTCTCT (SEQ ID NO: 164). In some embodiments, the second primer comprises the sequence CCATCTCATCCCTGCG (SEQ ID NO: 165). In some embodiments, the second primer comprises and/or consists of the sequence CCACTACGCCTCCGCTTTCCTCTCTATGGGCAGTCGGTGAT (SEQ ID NO: 166). In some embodiments, the second primer comprises and/or consists of the sequence CCATCTCATCCCTGCGTGTCTCCGACTCAG (SEQ ID NO: 167). In some embodiments, the second primer comprises and/or consists of the sequence CCACTACGCCTCCGCTTTCCTCTCTATGGGCAGTCGGTGA (SEQ ID NO: 168). In some embodiments, the second primer comprises and/or consists of the sequence CCATCTCATCCCTGCGTGTCTCCGACTCA (SEQ ID NO: 169). In some embodiments, the second primer comprises and/or consists of the sequence CCACTACGCCTCCGCTTTCCTCTCTATGGGCAGTCGGTG (SEQ ID NO: 170). In some embodiments, the second primer comprises and/or consists of the sequence CCATCTCATCCCTGCGTGTCTCCGACTC (SEQ ID NO: 171). In some embodiments, the second primer comprises and/or consists of the sequence CCACTACGCCTCCGCTTTCCTCTCTATGGGCAGTCGGT (SEQ ID NO: 172). In some embodiments, the second primer comprises and/or consists of the sequence CCATCTCATCCCTGCGTGTCTCCGACT (SEQ ID NO: 173). In some embodiments, the second primer comprises and/or consists of the sequence CCACTACGCCTCCGCTTTCCTCTCTATGGGCAGTCGG (SEQ ID NO: 174). In some embodiments, the second primer comprises and/or consists of the sequence CCATCTCATCCCTGCGTGTCTCCGAC (SEQ ID NO: 175). In some embodiments, the second primer comprises an indexing sequence. In some embodiments, the indexing sequence is a barcode sequence.

As used herein an “indexing sequence” and a “barcoding sequence” are used interchangeably and refer to a group of nucleotides that uniquely identify PCR products produced by a particular primer. Indexing sequences allow for multiple samples to be analyzed on the same sequencing run as the source of the product can be identified by the unique barcode/index. The index/barcode can be of any length that allows for unique identification such as 3, 4, 5, 6, 7, 8, 9 or 10 nucleotides. In some embodiments, an index is 5 nucleotides. In some embodiments, an index is 6 nucleotides. In some embodiments, an index is between 5-10 nucleotides. Any sequence may be used as its position within the second adapter will indicate that it is index sequence and not sequence from the sample. Sequences that do not occur naturally or are uncommon in nature may be used. In some embodiments, an index sequence is 3′ to a region that is bound by a sequencing primer.

Barcodes and adapters for inserting barcodes are commercially available. Examples of barcoding kits include, but are not limited to, barcodes for Ion Torrent platforms, such as KAPA Adapter Kits, and indexes for Illumina platforms, such as the TruSeq, TruSight and Nextera Kits.

As used herein, a “sequencing primer” is that primer used during deep sequencing or next generation sequencing analysis of a sample. This universal primer binds to all nucleic acid molecules in a sample and amplifies them during the sequencing reaction. And example of this is sequencing-by-synthesis, in which the sequencing primer is a start of an elongating new molecule that has labeled nucleotides incorporated that allow for sequencing. The index will thus be 3′ to the region to which the sequencing primer hybridizes, so that the index will be sequenced during the sequencing reaction. In some embodiments, an index sequence is directly 3′ to the region that is bound by a sequencing primer. In some embodiments, an index is 1, 2, 3, 4, 5, 6, 7, 8, 9, 10, 11, 12, 13, 14, 15, 16, 17, 18, 19 or 20 nucleotides downstream of the region that is bound by a sequencing primer. Each possibility represents a separate embodiment of the invention. In some embodiments, a sequencing primer comprises and/or consists of the sequence CAAGCAGAAGACGGCATACGAGAT (SEQ ID NO: 13).

In some embodiments, the second adapter sequence comprises and/or consist of the sequence AATGATACGGCGACCACCGAGATCTACACTCTT (SEQ ID NO: 10). In some embodiments, the second adapter sequence comprises and/or consist of the sequence CAAGCAGAAGACGGCATACGAGATGTGACTGG (SEQ ID NO: 11). In some embodiments, the second adapter sequence comprises and/or consist of the sequence CAAGCAGAAGACGGCATACGAGATNNNNNNGTGACTGG (SEQ ID NO: 12). In some embodiments, the second adapter sequence comprises and/or consist of the sequence CCACTACGCCTCCGCTTTCCTC (SEQ ID NO: 180). In some embodiments, the second adapter sequence comprises and/or consist of the sequence CCACTACGCCTCCGCTTTCCTCT (SEQ ID NO: 179). In some embodiments, the second adapter sequence comprises and/or consist of the sequence CCACTACGCCTCCGCTTTCCTCTC (SEQ ID NO: 178). In some embodiments, the second adapter sequence comprises and/or consist of the sequence CCACTACGCCTCCGCTTTCCTCTCT (SEQ ID NO: 177). In some embodiments, the second adapter sequence comprises and/or consist of the sequence CCACTACGCCTCCGCTTTCCTCTCTAT (SEQ ID NO: 176). In some embodiments, the second adapter sequence comprises and/or consist of the sequence CCATCTCATCCC (SEQ ID NO: 181). In some embodiments, the second adapter sequence comprises and/or consist of the sequence CCATCTCATCCCT (SEQ ID NO: 182). In some embodiments, the second adapter sequence comprises and/or consist of the sequence CCATCTCATCCCTG (SEQ ID NO: 183). In some embodiments, the second adapter sequence comprises and/or consist of the sequence CCATCTCATCCCTGC (SEQ ID NO: 184). In some embodiments, the second adapter sequence comprises and/or consist of the sequence CCATCTCATCCCTGCG (SEQ ID NO: 185). In some embodiments, the second adapter sequence comprises a region of homology to a sequencing primer. In some embodiments, the second adapter sequence comprises a region that hybridizes to a sequencing primer.

In some embodiments, the sequence of the second primer combined with the second region of the first adapter sequence is a sequencing adapter. In some embodiments, the sequence of the second primer combined with the second region of the first adapter sequence is a Truseq adapter. In some embodiments, the sequence of the second primer combined with the second region of the first adapter sequence is the Truseq universal adapter. In some embodiments, the sequence of the second primer combined with the second region of the first adapter sequence is the Truseq indexed adapter. In some embodiments, the sequence of the second primer combined with the second region of the first adapter sequence is the Ion Torrent P1 adapter. In some embodiments, the sequence of the second primer combined with the second region of the first adapter sequence is the Ion Torrent A adapter. In some embodiments, the sequence of the second primer combined with the second region of the first adapter sequence comprises and/or consists of the sequence AATGATACGGCGACCACCGAGATCTACACTCTTTCCCTACACGACGCTCTTCCG ATCT (SEQ ID NO: 14). In some embodiments, the sequence of the second primer combined with the second region of the first adapter sequence comprises and/or consists of the sequence CAAGCAGAAGACGGCATACGAGATGTGACTGGAGTTCAGACGTGTGCTCTTCC GATC (SEQ ID NO: 15). In some embodiments, the sequence of the second primer combined with the second region of the first adapter sequence comprises and/or consists of the sequence CAAGCAGAAGACGGCATACGAGA GTGACTGGAGTTCAGACGTGTG CTCTTCCGATC (SEQ ID NO: 16). In some embodiments, the sequence of the second primer combined with the second region of the first adapter sequence comprises and/or consists of the sequence CCACTACGCCTCCGCTTTCCTCTCTATGGGCAGTCGGTGAT (SEQ ID NO: 166). In some embodiments, the sequence of the second primer combined with the second region of the first adapter sequence comprises and/or consists of the sequence CCATCTCATCCCTGCGTGTCTCCGACTCAG (SEQ ID NO: 167).

In some embodiments, the primer set further comprises a third primer. In some embodiments, the third primer comprises a 3′ region of homology to a segment of the target molecule. In some embodiments, region of homology is the reverse compliment of the segment of the target molecule. In some embodiments, the segment is a 3′ segment of the target molecule. In some embodiments, the segment of the target molecule with homology to the third primer is 3′ to the segment with homology to the first primer. In some embodiments, the third primer is suitable for amplifying the target nucleic acid molecule in combination with the first primer. In some embodiments, the amplification is PCR. In some embodiments, the 3′ region of homology of the first primer and the third primer are a primer pair. In some embodiments, the primer pair was designed for amplification of at least a portion target nucleic acid molecule. In some embodiments, the primer pair was designed for amplification of the target nucleic acid molecule. Non-limiting examples of primer pairs for amplification of specific targets are provided in Table 1. It will be understood by a skilled artisan, that primers of a primer pair should be spaced sufficiently far apart as to amplify the desired locus efficiently. Primers placed to close and/or too far apart may result in inefficient amplification. Primer selection is well known in the art and can be performed with websites such as bioinfo.ut.ee/primer3-0.4.0/or pga.mgh.harvard.edu/primerbank/for example.

In some embodiments, the third primer further comprises a 5′ third adapter sequence. In some embodiments, the adapter sequence is directly 5′ to the region of homology. In some embodiments, there is a linker between the adapter sequence and the region of homology. In some embodiments, the third adapter sequence comprises a first and second region. In some embodiments, a third adapter has the same requirements and restrictions as a first adapter.

In some embodiments, the third adapter sequence comprises or consists of at least 10, 12, 15, 17, 20, 22, 25, 27 or 30 nucleotides. Each possibility represents a separate embodiment of the invention. In some embodiments, the third adapter sequence comprises or consists of between 10-35, 10-30, 10-27, 10-25, 10-22, 10-20, 12-35, 12-30, 12-27, 12-25, 12-22, 12-20, 15-35, 15-30, 15-27, 15-25, 15-22, 15-20, 17-35, 17-30, 17-27, 17-25, 17-22, 17-20, 20-35, 20-30, 20-27, 20-25, 20-22, 22-35, 22-30, 22-27, 22-25, 24-35, 24-30, 24-27, 24-26, 24-25, 25-35, 25-30, 25-27, or 25-26 nucleotides in length. Each possibility represents a separate embodiment of the invention. In some embodiments, the third adapter sequence comprises or consists of between 24-26 nucleotides. In some embodiments, the third adapter sequence consists of 25 nucleotides.

In some embodiments, the first region of the third adapter sequence is 5′ to the second region of the third adapter sequence. In some embodiments, the first region is the 5′ end of the third adapter sequence. In some embodiments, the second region is the 3′ end of the third adapter sequence. In some embodiments, the first region is larger than the second region. In some embodiments, the first region is larger than or equal to the second region. In some embodiments, the first region is 10-99, 20-99, 30-99, 40-99, 50-99, 60-99, 65-99, 10-95, 20-95, 30-95, 40-95, 50-95, 60-95, 65-95, 10-90, 20-90, 30-90, 40-90, 50-90, 60-90, 65-90, 10-85, 20-85, 30-85, 40-85, 50-85, 60-85, 65-85, 10-80, 20-80, 30-80, 40-80, 50-80, 60-80, 65-80, 10-75, 20-75, 30-75, 40-75, 50-75, 60-75, 65-75, 10-70, 20-70, 30-70, 40-70, 50-70, 60-70, or 65-70% of the third adapter sequence. Each possibility represents a separate embodiment of the invention. In some embodiments, the first region is 50-99% of the third adapter sequence.

In some embodiments, the first region of the third adapter comprises at least 3, 5, 7, 9, 10, 12, 14, 15, or 17 nucleotides. Each possibility represents a separate embodiment of the invention. In some embodiments, the first region of the third adapter comprises at most 17, 19, 20, 21, 23, 25, 27, 29, 30, 32, 34 or 35 nucleotides. Each possibility represents a separate embodiment of the invention. In some embodiments, the first region of the third adapter comprises between 5-30, 5-27, 5-25, 5-23, 5-20, 5-19, 5-18, 5-17, 7-30, 7-27, 7-25, 7-23, 7-20, 7-19, 7-18, 7-17, 10-30, 10-27, 10-25, 10-23, 10-20, 10-19, 10-18, 10-17, 12-30, 12-27, 12-25, 12-23, 12-20, 12-19, 12-18, 12-17, 14-30, 14-27, 14-25, 14-23, 14-20, 14-19, 14-18, 14-17, 15-30, 15-27, 15-25, 15-23, 15-20, 15-19, 15-18, 15-17, 16-30, 16-27, 16-25, 16-23, 16-20, 16-19, 16-18, or 16-17 nucleotides. Each possibility represents a separate embodiment of the invention. In some embodiments, the first region of the third adapter comprises 15-18 nucleotides. In some embodiments, the first region of the third adapter comprises 16 or 17 nucleotides.

In some embodiments, the second region of the third adapter sequence is 3′ to the first region of the third adapter sequence. In some embodiments, the second region of the third adapter is smaller than the first region of the third adapter. In some embodiments, the second region of the third adapter is smaller than or equal to the first region of the third adapter. In some embodiments, the second region is 1-50, 1-40, 1-30, 1-20, 1-10, 1-5, 5-50, 5-40, 5-30, 5-20, 5-10, 10-50, 10-40, 10-30, 10-20, 15-50, 15-40, 15-30, 15-20, 20-50, 20-40, 20-30, 25-50, 25-40 or 25-30% of the third adapter sequence. Each possibility represents a separate embodiment of the invention. In some embodiments, the second region is 1-50% of the third adapter sequence.

In some embodiments, the second region of the third adapter comprises at least 1, 3, 5, 7, 8, 9, 10, 11, 12, 14, 15, or 17 nucleotides. Each possibility represents a separate embodiment of the invention. In some embodiments, the second region of the third adapter comprises at most 8, 9, 10, 11, 12, 15, 17, 19, 20, 21, 23 or 25 nucleotides. Each possibility represents a separate embodiment of the invention. In some embodiments, the second region of the third adapter comprises between 1-15, 1-13, 1-10, 1-9, 1-8, 2-15, 2-13, 2-10, 2-9, 2-8, 3-15, 3-13, 3-11, 3-10, 3-9, 3-8, 5-15, 5-13, 5-10, 5-9, 5-8, 6-15, 6-13, 6-10, 6-9, 6-8, 7-15, 7-13, 7-10, 7-9, 7-8, 8-15, 8-13, 8-10 or 8-9 nucleotides. Each possibility represents a separate embodiment of the invention. In some embodiments, the second region of the third adapter comprises 6-10 nucleotides. In some embodiments, the second region of the third adapter comprises 8 nucleotides.

In some embodiments, the third adapter comprises and/or consists of the sequence of SEQ ID NO: 1. In some embodiments, the third adapter comprises and/or consists of the sequence of SEQ ID NO: 2. In some embodiments, the first region of the third adapter comprises and/or consists of the sequence of SEQ ID NO: 3. In some embodiments, the second region of the third adapter comprises and/or consists of the sequence of SEQ ID NO: 4. In some embodiments, the first region of the third adapter comprises and/or consists of the sequence of SEQ ID NO: 5. In some embodiments, the second region of the third adapter comprises and/or consists of the sequence of SEQ ID NO: 6.

In some embodiments, the fourth primer comprises a region similar to the first region of the third adapter. In some embodiment similar is identical. In some embodiments, similar is homologous. In some embodiments, similar is complementary. In some embodiments, a similar region comprises at most 1, 2, 3, 4, 5, 6, or 7 mismatches. In some embodiments, a similar region is identical to the first region of the third adapter. In some embodiments, the region similar to a first region of the third adapter is 3′ to a fourth adapter sequence. In some embodiments, the region similar to a first region of the third adapter is at the 3′ end of the fourth primer. In some embodiments, a region similar to the first region of the third adapter comprises and/or consists of SEQ ID NO: 3 or SEQ ID NO: 5. In some embodiments, a region similar to the first region of the third adapter is selected from SEQ ID NO: 3 and SEQ ID NO: 5.

In some embodiments, the fourth primer further comprises a fourth adapter sequence. In some embodiments, the fourth adapter sequence is 5′ to the similar region. In some embodiments, the fourth adapter sequence is the 5′ end of the second primer. In some embodiments, the fourth adapter sequence is a universal sequence. In some embodiments, the fourth adapter sequence comprises a primer sequence for a sequencing primer. In some embodiments, the primer sequence is complementary to a sequencing primer. In some embodiments, the fourth adapter sequence comprises at least 10, 15, 20, 25, 30, or 35 nucleotides. Each possibility represents a separate embodiment of the invention. In some embodiments, the fourth adapter sequence comprises at most 30, 35, 40, 45, 50, 55, or 60 nucleotides. Each possibility represents a separate embodiment of the invention. In some embodiments, the fourth adapter sequence comprises between 20-50, 25-50, 30-50, 31-50, 32-50, 33-50, 35-50, 20-45, 25-45, 30-45, 31-45, 32-45, 33-45, 35-45, 20-40, 25-40, 30-40, 31-40, 32-40, 33-40, 35-40, 20-35, 25-35, 30-35, 31-35, 32-35, 33-35, 20-34, 25-34, 30-34, 31-34, 32-34, or 33-34 nucleotides. Each possibility represents a separate embodiment of the invention.

In some embodiments, the fourth primer comprises and/or consists of the sequence of SEQ ID NO: 7. In some embodiments, the fourth primer comprises and/or consists of the sequence of SEQ ID NO: 9. In some embodiments, the fourth primer comprises an indexing sequence. In some embodiments, the indexing sequence is a barcode sequence. In some embodiments, the fourth primer comprises and/or consists of the sequence of SEQ ID NO: 8. In some embodiments, the barcode/index sequence is a unique six nucleotide sequence that identifies PCR products produced by the fourth primer.

In some embodiments, the fourth adapter sequence comprises and/or consist of the sequence of SEQ ID NO: 10. In some embodiments, the fourth adapter sequence comprises and/or consist of the sequence of SEQ ID NO: 11. In some embodiments, the fourth adapter sequence comprises and/or consist of the sequence of SEQ ID NO: 12. In some embodiments, the fourth adapter sequence comprises a region of homology to a sequencing primer. In some embodiments, the fourth adapter sequence comprises a region that hybridizes to a sequencing primer.

In some embodiments, the sequence of the fourth primer combined with the second region of the third adapter sequence is a sequencing adapter. In some embodiments, the sequence of the fourth primer combined with the second region of the third adapter sequence is a Truseq adapter. In some embodiments, the sequence of the fourth primer combined with the second region of the third adapter sequence is the Truseq universal adapter. In some embodiments, the sequence of the fourth primer combined with the second region of the third adapter sequence is the Truseq indexed adapter. In some embodiments, the sequence of the fourth primer combined with the second region of the third adapter sequence is the Ion Torrent P1 adapter. In some embodiments, the sequence of the fourth primer combined with the second region of the third adapter sequence is the Ion Torrent A adapter. In some embodiments, the sequence of the fourth primer combined with the second region of the third adapter sequence comprises and/or consists of the sequence of SEQ ID NO: 14. In some embodiments, the sequence of the fourth primer combined with the second region of the third adapter sequence comprises and/or consists of the sequence of SEQ ID NO: 15. In some embodiments, the sequence of the fourth primer combined with the second region of the third adapter sequence comprises and/or consists of the sequence of SEQ ID NO: 16. In some embodiments, the sequence of the fourth primer combined with the second region of the third adapter sequence comprises and/or consists of the sequence CCACTACGCCTCCGCTTTCCTCTCTATGGGCAGTCGGTGAT (SEQ ID NO: 166) or CCATCTCATCCCTGCGTGTCTCCGACTCAG (SEQ ID NO: 167).

In some embodiments, the second region of the first adapter sequence and the second region of the third adapter sequence are at least 50, 60, 70, 75, 80, 85, 90, 95, 99 or 100% identical. Each possibility represents a separate embodiment of the invention. In some embodiments, the second region of the first adapter sequence and the second region of the third adapter sequence are at least 85% identical. In some embodiments, the second region of the first adapter sequence and the second region of the third adapter sequence are identical save for 1, 2, 3, 4, or 5 mismatches or missing nucleotides. Each possibility represents a separate embodiment of the invention. In some embodiments, the second region of the first adapter sequence and the second region of the third adapter sequence are identical save for 1 mismatch or missing nucleotide. In some embodiments, the second region of the first adapter sequence and the second region of the third adapter sequence comprise a region that is identical and comprises at least 5, 6, 7, or 8 nucleotides. Each possibility represents a separate embodiment of the invention. In some embodiments, the second region of the first adapter sequence and the second region of the third adapter sequence comprise a region that is identical and comprises at least 8 nucleotides. In some embodiments, the region that is identical comprises the sequence TTCCGATC (SEQ ID NO: 17).

In some embodiments, the 3′ region of similarity of the second primer and/or the fourth primer is less than 50, 45, 40, 37, 36, 35, 34, 33, or 32% of the second and/or fourth primer. Each possibility represents a separate embodiment of the invention. In some embodiments, the 3′ region of similarity of the second primer and/or the fourth primer is less than 35% of the second and/or fourth primer. In some embodiments, the 3′ region of similarity of the second primer is less than 35% of the second primer. In some embodiments, the 3′ region of similarity of the fourth primer is less than 35% of the fourth primer.

In some embodiments, the second and/or fourth primers comprise at least 30, 35, 40, 45, 46, 47, 48, 49, or 50 nucleotides in length. Each possibility represents a separate embodiment of the invention. In some embodiments, the second and/or fourth primers comprise at most 49, 50, 51, 52, 53, 54, 55, 60, 65, or 70 nucleotides in length. Each possibility represents a separate embodiment of the invention. In some embodiments, the second and/or fourth primers comprise between 30-65, 35-65, 40-65, 45-65, 46-65, 47-65, 48-65, 49-65, 30-60, 35-60, 40-60, 45-60, 46-60, 47-60, 48-60, 49-60, 30-55, 35-55, 40-55, 45-55, 46-55, 47-55, 48-55, 49-55, 30-53, 35-53, 40-53, 45-53, 46-53, 47-53, 48-53, 49-53, 30-52, 35-52, 40-52, 45-52, 46-52, 47-52, 48-52, 49-52, 30-51, 35-51, 40-51, 45-51, 46-51, 47-51, 48-51, 49-51, 30-50, 35-50, 40-50, 45-50, 46-50, 47-50, 48-50, or 49-50 nucleotides in length. Each possibility represents a separate embodiment of the invention. In some embodiments, the second and/or fourth primers comprise between 45-60 nucleotides in length.

By another aspect, there is provided a kit comprising at least two primer sets of the invention.

In some embodiments, the kit comprises a plurality of primer sets of the invention. In some embodiments, at least two of the sets amplify different target nucleic acid molecules. In some embodiments, each first primer comprises a 3′ region of similarity to a different target nucleic acid molecule. In some embodiments, at least two first primers comprise 3′ regions of similarity to different target nucleic acid molecules. In some embodiments, at least two first primers comprise 3′ regions of similarity to different segments of a nucleic acid molecule. In some embodiments, the second adapter sequence is a universal sequence. In some embodiments, a universal sequence is shared by all primers. In some embodiments, a universal sequence is shared by all second primers. In some embodiments, the fourth adapter sequence is a universal sequence. In some embodiments, a universal sequence is shared by all fourth primers. In some embodiments, the second adapter sequence is common to all second primers. In some embodiments, the fourth adapter sequence is common to all fourth primers. In some embodiments, the first adapter sequence is common to all first primers. In some embodiments, the third adapter sequence is common to all third primers. In some embodiments, the first region of the first adapter is common to all first primers. In some embodiments, the first region of the third adapter is common to all third primers.

By another aspect, there is provided a method of PCR, the method comprising:
a. providing a sample comprising a target nucleic acid molecule;
b. performing a first PCR reaction with the target nucleic acid molecule and a first primer of a primer set of the invention to produce a first adapter labeled target nucleic acid hybrid molecule; and
c. performing a second PCR reaction with the hybrid molecule and a second primer of a primer set of the invention to produce a first and second adapter labeled target nucleic acid hybrid molecule;

thereby performing PCR.

By another aspect, there is provided a method of multiplex PCR, the method comprising:
a. providing a sample comprising at least two target nucleic acid molecules;
b. performing a first PCR reaction with at the at least two target nucleic acid molecules and at least two first primers of a primer set of the invention to produce first adapter labeled target nucleic acid hybrid molecules, wherein the at least two first primers hybridize to different targets and comprise identical first region of a first adapter; and
c. performing a second PCR reaction with the hybrid molecules and a second primer of the primer set of the invention to produce first and second adapter labeled target nucleic acid hybrid molecules;

thereby performing multiplex PCR.

In some embodiments, the multiplex PCR is generation of a sequencing library. In some embodiments, the multiplex PCR is amplification of at least 2, 3, 4, 5, 10, 15, 20, 25, 30, 35, 40, 45 or 50 target molecules in one multiplex PCR. Each possibility represents a separate embodiment of the invention. In some embodiments, the method further comprises sequencing the first and second adapter labeled target nucleic acid hybrid molecules. In some embodiments, the sequencing is with a sequencing primer and the second adapter comprises a sequence of the sequencing primer.

In some embodiments, the different targets are different nucleic acid molecules. In some embodiments, different targets are different segments of the same target molecule. In some embodiments, the different targets are different genomic loci. In some embodiments, the different targets are different methylation loci. In some embodiments, the different targets result in amplification of different loci. In some embodiments, the at least two target nucleic acid molecule are a first and second strand of a target double stranded nucleic acid molecule. In some embodiments, the double stranded molecule is a bisulfite converted double stranded molecule.

In some embodiments, the sample comprises cfDNA. In some embodiments, the sample comprises circulating tumor DNA (ctDNA). In some embodiments, cfDNA is ctDNA. In some embodiments, the sample is a bodily fluid sample. In some embodiments, the bodily fluid is blood. In some embodiments, the bodily fluid is selected from blood, plasma, urine, feces, cerebral-spinal fluid, semen, vaginal fluid, breast milk, sweat, and tears. In some embodiments, the method further comprises extracting nucleic acid molecules from the sample. In some embodiments, the sample comprises bisulfite converted nucleic acids. In some embodiments, nucleic acids from the sample have undergone bisulfite conversion.

In some embodiments, the first PCR reaction comprises the conditions described herein in the Methods section. In some embodiments, the second PCR reaction comprises the conditions described herein in the Methods section. In some embodiments, the first PCR reaction comprises at least 10, 15, 17, 18, 19, 20, 21, 22, 23 or 25 cycles. Each possibility represents a separate embodiment of the invention. In some embodiments, the first PCR reaction comprises at most 15, 20, 22, 25, 30 m 35, 40, 45 or 50 cycles. Each possibility represents a separate embodiment of the invention. In some embodiments, the first PCR reaction comprises 10-40, 10-35, 10-30, 10-25, 10-22, 10-20, 15-40. 15-35, 15-30, 15-25, 15-22, 15-20, 17-40, 17-35, 17-30, 17-25, 17-22, 17-20, 20-40, 20-35, 20-30, 20-25 or 20-22 cycles. Each possibility represents a separate embodiment of the invention. In some embodiments, the first PCR comprises 20 cycles. In some embodiments, the first PCR reaction is a gradient PCR. In some embodiments, the gradient PCR increases in annealing temperature as the PCR progresses. In some embodiments, the gradient PCR increases at the beginning of the reaction and then stabilizes at an annealing temperature for the rest of the run. A skilled artisan will appreciate that rounds of PCR where a first (or third) primer of the invention binds to an original target molecule will require a lower temperature as only the region of similarity is binding. Subsequent cycles where the adapter also can bind to a hybrid adapter labeled molecule will perform better at higher temperatures as a longer stretch of nucleotides must bind.

In some embodiments, the first PCR reaction comprises a third primer of the invention. In some embodiments, the second PCR reaction comprises a fourth primer of the invention. In some embodiments, the first and third primer are a primer pair that amplifies the target nucleic acid molecule and produce a first and third adapter labeled target hybrid nucleic acid molecule. In some embodiments, the second and fourth primer are a primer pair and amplify the first and third adapter labeled target hybrid nucleic acid molecule to produce a first, second, third and fourth adapter labeled target hybrid nucleic acid molecule. In some embodiments, the method of multiplex PCR comprises a universal second primer for amplifying all first adapter labeled target nucleic acid hybrid molecules. In some embodiments, the method of multiplex PCR comprises a universal fourth primer for amplifying all third adapter labeled target nucleic acid hybrid molecules. In some embodiments, the second and fourth primers are a universal primer pair.

In some embodiments, the method is performed in vitro. In some embodiments, the method is performed ex vivo. In some embodiments, the method is performed in culture. In some embodiments, the method is performed in vivo.

By another aspect, there is provided a method of determining the cell type of origin of a target nucleic acid, the method comprising:
a. providing a sample comprising at least two target nucleic acid molecules, that comprise at least one cell type-specific methylation/unmethylated site and wherein the at least two target nucleic acid molecules have undergone bisulfate conversion;
b. performing a first PCR reaction with the at least two target nucleic acid molecules and at least two first primers of the invention to produce first adapter labeled target nucleic acid hybrid molecules, wherein each of the first primers comprises
   i. a 3′ region of homology to a 5′ segment of one of the at least two target nucleic acid molecules; and
   ii. a 5′ first adapter sequence common to all first primers wherein the first adapter sequence comprises a 5′ first and 3′ second region;
c. performing a second PCR reaction with the first primer labeled hybrid molecules and at least two second primers to produce first and second adapter labeled target nucleic acid hybrid molecules, wherein each of the second primers comprises
   i. a 3′ region identical to the first region of the first adapter sequence; and
   ii. a 5′ second adapter sequence;
d. sequencing the first and second adapter labeled target nucleic acid molecules; and
e. determining a methylation status of the methylation/unmethylated site according to the base sequenced at the methylation/unmethylated site;
   wherein the presence of a methylation mark or lack of a methylation mark that is cell type-specific indicates that the target nucleic acid originates from the cell type, thereby determining the cell type of origin of the target nucleic acid.

In some embodiments, the at least two target nucleic acid molecules are cfDNA. In some embodiments, the first and second primers are primers of the invention. In some embodiments, two pairs of primer of the invention are used. In some embodiments, the two pairs share a common second primer. In some embodiments, the first PCR reaction uses a third primer of the invention. In some embodiments, each first primer has a third primer of the invention that is a primer pair for amplifying a target sequence comprising at least one cell type-specific methylation/unmethylated site. In some embodiments, the second PCR reaction uses a fourth primer of the invention. In some embodiments, a common fourth primer is used for all amplifications.

In some embodiments, the first PCR is performed with at least 2, 3, 4, 5, 6, 7, 8, 9, 10, 15, 20, 25, or 30 first primers. Each possibility represents a separate embodiment of the invention. In some embodiments, each first primer comprises a region of similarity to a target nucleic acid molecule with a cell type-specific methylation/unmethylated site. In some embodiments, each cell type-specific methylation/unmethylated site is for a different cell type. In some embodiments, at least 2, 3, 4, 5, 6, 7, 8, 9 or 10 different cell type-specific methylation/unmethylated sites are all informative for the same cell type. Each possibility represents a separate embodiment of the invention.

As used herein, a “cell type-specific methylation/unmethylated site” refers to at least one cytosine whose methylation of unmethylation status is mostly unique or specific to a specific cell type. That is having a methylation at this particular cytosine may be specific to a cell type, such that all other cell types or most other cell types do not have methylation at this particular cytosine. Similarly lack of a methylation at this particular cytosine may be specific to a cell type, such that all other cell types or most other cell types have a methylation at this particular cytosine. It may also be that a group of cytosines in close proximity have a particular pattern that is cell type specific. Thus, the site may be more than one cytosine, and the specificity may be in the pattern of methylation. For example, if there are three cytosines they may be methylated, unmethylated, unmethylated in a particular tissue while all other, or most other, tissues have a different pattern.

In some embodiments, a cell type is a tissue. In some embodiments, unique methylation/unmethylation comprises at most 30, 25, 20, 15, 10, 5, 3, 2 or 1% methylation/unmethylation in cell types that are not the specific cell type. In some embodiments, unique methylation/unmethylation comprises at least 60, 65, 70, 75, 80, 85, 90, 95, 97, 99 or 100% methylation/unmethylation in the specific cell type. In some embodiments, unique methylation/unmethylation comprises at least 80% methylation in the specific cell type and less than 30% methylation/unmethylation in all other cell types. Cell type specific methylation marks are well known in the art and can be found in numerous academic articles, such as for non-limiting example Moss et al., 2018, Comprehensive human cell-type methylation atlas reveals origins of circulating cell-free DNA in health and disease, Nature Communications, 9(1):5068, herein incorporated by reference in its entirety. By selecting tissue-specific loci from a methylome atlas and then employing the method of the invention to determine the methylation status at these loci, the origin of each target molecule can be determined.

In some embodiments, the first PCR is performed with a first first-primer with homology to a forward strand of a target nucleic acid molecule with a cell type-specific methylation/unmethylated site and a second first primer with homology to a reverse strand of the same nucleic acid molecule. In some embodiments, only one strand has a cell type-specific methylation/unmethylated site. In some embodiments, both strands have cell type-specific methylation/unmethylated site. In some embodiments, to increase accuracy at least one first primer that hybridizes to an opposite strand of a locus that is probed by another first primer is used in the first PCR.

In some embodiments, the sample is from a subject. In some embodiments, the subject is at risk for cell death of a target cell type. In some embodiments, the subject is a healthy subject. In some embodiments, the subject is a subject suffering from a disease. In some embodiments, the subject has undergone surgery. In some embodiments, the subject has undergone a transplant. In some embodiments, the transplant is an islet transplant. In some embodiments, the method is for determining cell death of cell of the cell type of origin. In some embodiments, the method is for determining cell death of a target cell type. In some embodiments, the cell death is within the subject. In some embodiments, the method is for determining cell death in the subject.

In some embodiments, determining a cell type of origin or detecting death of a cell type comprises detection of at least 1, 2, 3, 4, 5, or 6 cell-type specific methylation loci. Each possibility represents a separate embodiment of the invention. In some embodiments, determining a cell type of origin or detecting death of a cell type comprises detection of at least 2 cell-type specific methylation loci. In some embodiments, determining a cell type of origin or detecting death of a cell type comprises detection of at least 3 cell-type specific methylation loci. In some embodiments, determining a cell type of origin or detecting death of a cell type comprises detection of at least 4 cell-type specific methylation loci. In some embodiments, determining a cell type of origin or detecting death of a cell type comprises detection of at least 5 cell-type specific methylation loci. In some embodiments, determining a cell type of origin or detecting death of a cell type comprises detection of at least 6 cell-type specific methylation loci.

By another aspect, there is provided a method of detecting cell free DNA (cfDNA) in a sample, the method comprising:
a. receiving a sample comprising cfDNA; and
b. detecting in the sample a DNA sequence of the insulin (INS) gene, wherein the region is between nucleotides 1058-1222 downstream of the INS transcriptional start site and/or comprises at least one cytosine base selected from cytosine 1080, 1102, 1116, 1124, 1170, 1173, 1181, 1195, 1197 and 1202;
   thereby detecting cfDNA in a sample.

According to another aspect, there is provided a method of detecting beta cell cfDNA in a sample, the method comprising:
a. receiving a sample comprising cfDNA;
b. detecting in the sample a DNA sequence of the insulin (INS) gene, wherein the region is between nucleotides 1058-1222 downstream of the INS transcriptional start site and/or comprises at least one cytosine base selected from cytosine 1080, 1102, 1116, 1124, 1170, 1173, 1181, 1195, 1197 and 1202; and
c. determining a methylation status of said at least one cytosine base, wherein absence of methylation on said at least one cytosine base indicates the presence of beta cell cfDNA;
   thereby detecting beta cell cfDNA in a sample.

In some embodiments, the cfDNA is unmethylated cfDNA. In some embodiments, the method is a method for detecting unmethylated cfDNA. In some embodiments, the cfDNA is unmethylated in the region of the INS gene. In some embodiments, the cfDNA is methylation sensitive converted cfDNA. In some embodiments, the cfDNA is methylation specific converted cfDNA. In some embodiments, converted cfDNA is cfDNA in which an unmethylated cytosine base is converted to a thymine base. In some embodiments, all unmethylated cytosine bases are converted to thymine bases. In some embodiments, methylated cytosine bases are not converted to thymine bases. In some embodiments, methylated cytosine bases remain cytosines. In some embodiments, the method of conversion is bisulfite conversion. In some embodiments, the converted cfDNA is cfDNA that has undergone bisulfite conversion. In some embodiments, the cfDNA is bisulfite converted cfDNA.

It will be understood that cfDNA that has undergone methylation sensitive conversion, for example bisulfite conversion, will require amplification with conversion specific primers. That is if PCR is used for amplification and sequencing the primers either will need not to include cytosines, or more likely to have a C to T converted sequence. For example, the primers provided in SEQ ID NO: 186-187 are for amplification of the entire INS10 region of the insulin gene after bisulfite conversion. Other primers can be designed for amplification of smaller portions of the INS10 region, or for amplification of any converted gene, so long as they are designed with the loss of cytosines in mind. Standard primer production software and techniques can be employed, such as, for example, Primer3.

In some embodiments, the sample is a blood sample. In some embodiments, the blood sample is a peripheral blood sample. In some embodiments, the sample is a tissue sample. In some embodiments, the sample is a serum sample. In some embodiments, the sample is a plasma sample. In some embodiments, the sample is a bodily fluid sample. In some embodiments, the bodily fluid is selected from blood, serum, plasma, urine, feces, cerebral spinal fluid, lymph, breast milk and amniotic fluid. In some embodiments, the sample is isolated and/or purified cfDNA.

In some embodiments, detecting comprises sequencing of the region. In some embodiments, sequencing is standard Sanger sequencing. In some embodiments, sequencing is Next Generation Sequencing. In some embodiments, detecting comprises PCR amplification of the region. In some embodiments, the PCR amplification is methylation specific PCR amplification. In some embodiments, the PCR amplification comprises primers that amplify at least a portion of the region. In some embodiments, the portion comprises at least one of the listed cytosine bases. In some embodiments, the PCR amplification is methylation specific and a primer of the PCR binds to a portion of the region comprising at least one of the listed cytosine bases.

In some embodiments, the region is between nucleotides 1058-1222 downstream of the transcriptional start site of the insulin gene. In some embodiments, the region comprises or consist of the nucleotide sequence TCCCTCTAACCTGGGTCCAGCCCGGCTGGAGATGGGTGGGAGTGCGACCTAGG GCTGGCGGGCAGGCGGGCACTGTGTCTCCCTGACTGTGTCCTCCTGTGTCCCTC TGCCTCGCCGCTGTTCCGGAACCTGCTCTGCGCGGCACGTCCTGGCAGTGGGG CAGGT (SEQ ID NO: 188). In some embodiments, the region is the entire region from nucleotides 1058-1222. In some embodiments, the region is a portion of the region from nucleotides 1058-1222. In some embodiments, the region is a portion of SEQ ID NO: 188. In some embodiments, the region comprises or consists of at least 10, 15, 20, 25, 30, 35, 40, 45, 50, 60, 70, 75, 80, 90, 100, 110, 120, 130, 140, 150, 160, or 164 nucleotides. Each possibility represents a separate embodiment of the invention.

In some embodiments, the region or portion of the region comprises as least one cytosine base. In some embodiments, the region or portion of the region comprises at least one CpG dinucleotide. In some embodiments, the region or portion of the region comprises at least one methylatable cytosine base. In some embodiments, the region comprises at least one cytosine base selected from cytosine 1080, 1102, 1116, 1124, 1170, 1173, 1181, 1195, 1197 and 1202 downstream of the transcriptional start site of the insulin gene. In some embodiments, the region comprises a plurality of cytosine bases. In some embodiments, the region or portion of the region comprises 1, 2, 3 4, 5, 6, 7, 8, 9 or 10 cytosine bases. Each possibility represents a separate embodiment of the invention. In some embodiments, the portion of the region comprises at least one cytosine base selected from cytosine 1080, 1102, 1116, 1124, 1170, 1173, 1181, 1195, 1197 and 1202 downstream of the transcriptional start site of the insulin gene.

In some embodiments, the sequence of the insulin gene comprises or consist of the nucleotide sequence AGCCCTCCAGGACAGGCTGCATCAGAAGAGGCCATCAAGCAGGTCTGTTCCAA GGGCCTTTGCGTCAGGTGGGCTCAGGATTCCAGGGTGGCTGGACCCCAGGCCC CAGCTCTGCAGCAGGGAGGACGTGGCTGGGCTCGTGAAGCATGTGGGGGTGA GCCCAGGGGCCCCAAGGCAGGGCACCTGGCCTTCAGCCTGCCTCAGCCCTGCC TGTCTCCCAGATCACTGTCCTTCTGCCATGGCCCTGTGGATGCGCCTCCTGCCC CTGCTGGCGCTGCTGGCCCTCTGGGGACCTGACCCAGCCGCAGCCTTTGTGAA CCAACACCTGTGCGGCTCACACCTGGTGGAAGCTCTCTACCTAGTGTGCGGGG AACGAGGCTTCTTCTACACACCCAAGACCCGCCGGGAGGCAGAGGACCTGCAG GGTGAGCCAACTGCCCATTGCTGCCCCTGGCCGCCCCCAGCCACCCCCTGCTCC TGGCGCTCCCACCCAGCATGGGCAGAAGGGGGCAGGAGGCTGCCACCCAGCA GGGGGTCAGGTGCACTTTTTTAAAAAGAAGTTCTCTTGGTCACGTCCTAAAAGT GACCAGCTCCCTGTGGCCCAGTCAGAATCTCAGCCTGAGGACGGTGTTGGCTT CGGCAGCCCCGAGATACATCAGAGGGTGGGCACGCTCCTCCCTCCACTCGCCC CTCAAACAAATGCCCCGCAGCCCATTTCTCCACCCTCATTTGATGACCGCAGAT TCAAGTGTTTTGTTAAGTAAAGTCCTGGGTGACCTGGGGTCACAGGGTGCCCC ACGCTGCCTGCCTCTGGGCGAACACCCCATCACGCCCGGAGGAGGGCGTGGCT GCCTGCCTGAGTGGGCCAGACCCCTGTCGCCAGGCCTCACGGCAGCTCCATAG TCAGGAGATGGGGAAGATGCTGGGGACAGGCCCTGGGGAGAAGTACTGGGAT CACCTGTTCAGGCTCCCACTGTGACGCTGCCCCGGGGCGGGGGAAGGAGGTGG GACATGTGGGCGTTGGGGCCTGTAGGTCCACACCCAGTGTGGGTGACCCTCCC TCTAACCTGGGTCCAGCCCGGCTGGAGATGGGTGGGAGTGCGACCTAGGGCTG GCGGGCAGGCGGGCACTGTGTCTCCCTGACTGTGTCCTCCTGTGTCCCTCTGCC TCGCCGCTGTTCCGGAACCTGCTCTGCGCGGCACGTCCTGGCAGTGGGGCAGG TGGAGCTGGGCGGGGGCCCTGGTGCAGGCAGCCTGCAGCCCTTGGCCCTGGAG GGGTCCCTGCAGAAGCGTGGCATTGTGGAACAATGCTGTACCAGCATCTGCTC CCTCTACCAGCTGGAGAACTACTGCAACTAGACGCAGCCCGCAGGCAGCCCCA CACCCGCCGCCTCCTGCACCGAGAGAGATGGAATAAAGCCCTTGAACCAGC (SEQ ID NO: 189). In some embodiments, the sequence of the region comprises or consist of the nucleotide sequence of SEQ ID NO: 188.

In some embodiments, the method further comprises determining the methylation status of the at least one cytosine base. In some embodiments, the method further comprises determining the methylation status of a plurality of cytosine bases in the region. In some embodiments, the method further comprises determining the methylation status of all the cytosines in the region. It will be understood by a skilled artisan that since the sequence of the region is known methylation status for any cytosine can be determined. For non-limiting example, is bisulfate conversion has been performed and the sequence of the cfDNA determined, any location where a thymine is determined in the sequence where there should be a cytosine indicates that that cytosine was unmethylated. Conversely, any cytosine of the cytosines listed, that when sequences are still cytosines and not thymines, then it indicates that the cytosine is methylated.

In some embodiments, the method is for detecting cfDNA in a sample. In some embodiments, the method is for detecting beta cell cfDNA in a sample. In some embodiments, the sample is from a subject. In some embodiments, the sample is from a subject in need of determining the presence of cfDNA. In some embodiments, the detecting beta cell cfDNA comprises detecting beta cell death in the subject. In some embodiments, the method is for detecting beta cell death in a subject. In some embodiments, beta cell death is indicative of a beta cell-associated pathology in the subject. In some embodiments, the method is for detecting a beta cell-associated pathology in the subject.

In some embodiments, the beta cell-associated pathology is a beta cell disorder, disease or condition. In some embodiments, the pathology is selected from pancreatic cancer, diabetes and hyperinsulinism. In some embodiments, the pathology is pancreatic cancer. In some embodiments, the pancreatic cancer is beta cell cancer. In some embodiments, the pathology is diabetes. In some embodiments, the pathology is hyperinsulinism. In some embodiments, the hyperinsulinism is congenital hyperinsulinism. In some embodiments, the hyperinsulinism is congenital hyperinsulinism of infancy. In some embodiments, a beta cell condition is an islet transplant. In some embodiments, the beta cell pathology is rejection of an islet transplant.

In some embodiments, the method further comprises administering a therapeutic agent that treats the pathology. In some embodiments, the therapeutic agent is insulin. In some embodiments, the therapeutic agent is an anti-cancer agent. In some embodiments, the therapeutic agent is a cytotoxic agent.

As used herein, the term “about” when combined with a value refers to plus and minus 10% of the reference value. For example, a length of about 1000 nanometers (nm) refers to a length of 1000 nm+-100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods:

### Sample Preparation and DNA Processing.

Blood samples were collected in 10 ml EDTA blood tubes or Streck® blood tubes and mixed by gentle inversion. Tubes were stored at room temperature. EDTA tubes were centrifuged within 4 hours and Streck tubes within 5 days. Blood tubes were centrifuged for 10 minutes at 1,500×g. EDTA tubes at 4° C. and Streck tubes at room temperature. The supernatant was transferred to a fresh 15 ml conical tube without disturbing the cellular layer and centrifuged again for 10 min at 3000×g. The supernatant was collected and stored in −80c.

Cell-free DNA was extracted from 1-4 mL of plasma using the QIAsymphony liquid handling robot (Qiagen). cfDNA was treated with bisulfite using EZ DNA Methylation-Gold™ (Zymo Research), according to the manufacturer's instructions. DNA concentration was determined using Qbit double-strand molecular probes (Invitrogen).

Islets from cadaveric donors were used to isolate β-cells, acinar cells and duct epithelial cells. Genomic DNA from other tissues was purchased from standard vendors.

DNA derived from all samples was treated with bisulfite using EZ DNA Methylation-Gold™ (Zymo Research), according to the manufacturer's instructions, and eluted in 20 μl elution buffer.

Tissue-specific methylation candidate biomarkers were selected using comparative methylome analysis, based on publicly available datasets. Candidate markers were defined as loci having more than five CpG sites within 150 bp, with an average methylation value less than 0.4 in the tissue of interest, greater than 0.9 in leukocytes and greater than 0.8 in over 90% of tissues. Alternatively, candidate markers were selected based on being methylated in the tissue of interest (greater than 0.8) but unmethylated (less than 0.3) in all other tissues including leukocytes.

Primers for differentially methylated areas were designed to amplify bisulfite-treated DNA. Amplification is independent of methylation status at the monitored CpG sites. Primers used herein are provided in Table 1.

TABLE 1
Primers for differentially methylated areas

| | Forward primer (SEQ ID NO:) | Reverse primer (SEQ ID NO:) |
|---|---|---|
| 509 | GTATGATTTTTTGGGGGTTAG (18) | CTTAACACCAACCTACTAAACTAAA (19) |
| AST1 | TGGATAGATATGGAAGTATGTGA (20) | AAAAAACCCTCATACACTCAA (21) |
| ATP11A | TTAAATGTAAAGTAATGTTTGTTTT (22) | AAATTCACAAACTCTAACTACTACAC (23) |
| C7orf50 | GGTTAGATTTTTATGGTAGTGAGG (24) | AAACTATTAACTAACACTAAACTCCC (25) |
| CD8A | TTAGTTTTTTTAGTATGATTTTGAG (26) | CACCACAAAAATCACAATACTAT (27) |
| CD8B | GTTAAGAAATTAATAGGAAAAAGAA (28) | AAAACCCCATATTACTTCCC (29) |
| cg00256155 | TGTGAATAGAAAAAGTTTTAAATATG (30) | CAAACCCTCCACCCTAACTTC (31) |
| CG0978 | TATATGTGTGTAGGTTGAATAAAAT (32) | TCCATTTCATATCAATACTAATATT (33) |
| cg27384476 | TAGGAGTAGGAATGGGGAGG (34) | CTCAATAATACTTCTCCTACCCAC (35) |
| COL1 | GTTTTTTGTTTTTGTAGGTTGA (36) | CTTCAAAATACAAAACACTCATCT (37) |
| CPA | GAGGAGGAGTAGGAGTAGATGTT (38) | GAGGAGGAGTAGGAGTAGATGTT (39) |
| DENND3 | GTTTGTTTTGAGATGTGAGAAT (40) | ATAACATCCTTACAAACTCACAA (41) |
| ECH1 | GTTAGAAGGTATAGAAATAATTGTTAT (42) | TCTCCAAACTCTAAAAACCCT (43) |
| endo7922 | AGGTTGTATGTTGAGTTAGATGTTAT (44) | CTATATAACCATACTATTAACCAACATT (45) |
| FAM101A | GTTTGGTAATTTATTTAGAGAAGTAA (46) | CCCACAAATAAAAAAAATACTC (47) |
| FAM101A RV | AAGAGTATTAGGAAAAGTGTAGGTT (48) | TATCCAAAAAAACAAAATAACC (49) |
| FAT1 | GTGTTGTTATTTAAGTTATTGAGAGTA (50) | AAATACCTCAAAAAACCTAAACTA (51) |
| FBXL19 | TTGGTAGGTTTGGAGTTGATAG (52) | AAAAATAAACACTAAAATCCCC (53) |
| FBXW8 | TTATATTTATTTTTAATTATATAGGGG (54) | GGTTTTCTGCTTCCTTTCAACCAG (55) |
| FGFGL1 | TTGTAGTGTTTAGTGGAGGGG (56) | CTACTTCACCTTCAACCCCTA (57) |
| FOXP3 | TTAGGTTTGGATTTTAATTTTG (58) | CCCTAACCCTTATCTACTCCA (59) |
| FOXP3TSDR | TGGGTTTTGTTGTTATAGTTTT (60) | ATATCTACCCTCTTCTCTTCCTC (61) |
| I | GTTATTAGTTTTTATTGTAGTTATTTTGA (62) | AATAACAAACAAACACACAAAACA (63) |
| IGF2R | TGGGTGTTGTTATTTTGTTGA (64) | CTACAAAAATACACACCCCAA (65) |
| INS | TTGTTGGTTTTTTGGGGATT (66) | ACCCTACAAATCCTCTACCTCC (67) |
| INS anti | GTTTTATTTTGTAGGTTTTTTGTTT (68) | CTACTAACCCTCTAAAAACCTAAAC (69) |
| ITF | TTAAGTTTGAAGGTGTAGGTTTT (70) | AAAACCCACACACACACACATAA (71) |
| ITIH4 | ATAGTGAAGATGTTAGTTTGTTTTT (72) | AACACACTTACCTAATAACCAAAC (73) |
| J | GTTAGAAGGTATAGAAATAATTGTTAT (74) | TCTCCAAACTCTAAAAACCCT (75) |
| KRT19 | GGTTTAGTAGTTAGGATAGGGTAG (76) | AAAAATAACCAAACCTAAACTATAC (77) |
| LENG8 | TAGGTTTTTTTAGTATAGTATGGTG (78) | CAACTCCTAACTTACTAATACTAACC (79) |
| LMX1B | GGAGGTTGGGAGAGGTTT (80) | CCAACTACAAACATCTACTTTAATAC (81) |
| LRP5 | GTTGTAGGTGTTTATTGGTATTG (82) | CCTAAATAAACAACTCCAAATAA (83) |
| LUAD1 | TTAGTTTTTTAGTTTATAGTTAGTATTAGT (84) | TCTCATCCAATACAAAAAAAATA (85) |
| MCF2L | GGGTAGGAGGAGTTGTTGGT (86) | ATACCCCCCATATCACAACTAC (87) |
| MONO1 | TTTGTTAGGTTAAGTAATTTGTAAA (88) | CATCTCCTACTTAAATAACTTCAAT (89) |
| MTG1 | GGAGGTTGTAGTGAGTTAAGATTA (90) | TCAACAACTACTCAATTACACACTA (91) |
| NAT10 | ATTTTTTTTGGTTGGATTGTT (92) | TCACAAACACACAAACCCAA (93) |
| NEUT | TTTTAAGAAGTTTTTGTGTTATTAT (94) | TCTAAAAATACCTAAATACAAACC (95) |
| NMR | ATGGAGTTTGAGTTATATAGGTATG (96) | AAACTCCTAAACTCAAAAAATCTA (97) |
| PAN4 | TTTATTTTATATTTAAGTTTAGGTGAT (98) | CCCACTTACATATATATAAACCTAAA (99) |
| PCYT1A | GGGGTATTTTTTATTATTTTATT (100) | ACACACAACTTCAAAAACTTCA (101) |
| PRDM2 | GAAAGAGGGGATAATAAATAGTT (102) | CTTATTACTATCACTCCCAAACTAA (103) |
| PRKCH | GGTGTTATAGGTAGGGTAGAGAA (104) | CCAACATTTATCATTTTCTTCA (105) |
| RAB4 | GGTTTTGGGAGGTTAGTGG (106) | TCACCAAAACTCCAACTTCA (107) |
| SLC | GAGAAGGTAATGTTGTTGGAAT (108) | ATAACACAAACATTAACTTCAAAC (109) |
| SNX1 | TTTTATGTATAGATTAATAGTAAAGTTTT (110) | AAACCAACATTTCTCTATAACTACT (111) |
| SORL1 | AGGTTGTTTTTTTATTTTTTAGAT (112) | TTTCCCTCCCTTTAATAACTAT (113) |
| SPATA13 | AGTATTTTTATTGGGTTGGAT (114) | CCTACTACCTCAAATTAACTAAAA (115) |
| TAF8 | AGTAGATGTATTATTGTGAAGAAGAA (116) | AACAATAAATAATAAATCTCTCAAAA (117) |
| TCFL2 | TGAAGGAAATGAGAGTAAAGGT (118) | CCCTTCTCCCTAAAAAAAAC (119) |
| TRPV1 | TTTTAAAGAAGTTTTTATGGGT (120) | ATAAACCAAACAACACTACACAT (121) |
| UBE | TTTAGTGTTAGAATTGAAAGAGTAGA (122) | TTAACCTTAACTATATCTAACAAAAA (123) |
| VTN2 | GGTATTTTGAAGAGGTAGGTTT (124) | ACCTAAATACCCCAAACTCAT (125) |
| WB1 | ATTTGATTTTGTGGTAGTGGA (126) | AAAATCCCCACCTCTACTTAA (127) |
| WOX | GTTTTTTGTTTTAGAATTGGTTA (128) | AATCCATAAATAAAACAAACCC (129) |
| ZC3H3 | GTTTTTTTATATATAATTATAAGTTGTT (130) | ATAAAAATACTTACTACTACCTTTCC (131) |
| ZFP | TTTGGTTTTGGTGTATTTTTT (132) | CAAAACCTACTATAATACTTCTAAACT (133) |
| ZNF238 | GTGGGGTAAGAGTTTTTAGTATT (134) | CCAATATATCTATACAAATCCCC (135) |
| ZNF296 | GGTTGAGTTTATTGTTTTGGG (136) | TTTACCAACAACAACAACCTAAC (137) |
| INS_INS10 | TTTTTTTAATTTGGGTTTAGTT (186) | ACCTACCCCACTACCAAAAC (187) |

### Selection of β-Cell Markers

β-cell-specific methylation candidate biomarkers were selected using comparative methylome analysis, based on publicly available datasets, to identify loci having more than five CpG sites within 150 bp, with an average methylation value for a specific cytosine (present on Illumina 450K arrays) of less than 0.4 in the β-cells, greater than 0.9 in leukocytes, and greater than 0.8 in over 90% of tissues. There were identified ˜200 CpG sites that are unmethylated in β-cells and methylated in all other major tissues, as well as ˜30 CpG sites that are methylated in β-cells and unmethylated elsewhere. Four of these sites (i.e., Fbx119, Mtgl, Leng8, and Zc3h3) were selected and primers were designed to amplify ˜100 bp fragments surrounding them using a novel multiplex two-step PCR amplification method (see below, and Table 1). In addition, primers to amplify a previously described fragment of the insulin gene were designed, separately targeting the sense and antisense strands of the insulin fragment, since each strand can serve as an independent marker in bisulfate-treated DNA, potentially increasing assay sensitivity (Table 1).

### PCR

1st step PCR: Multiplex PCR-sequencing assays were developed to increase the sensitivity and specificity of our assay using up to 30 pairs of primers in one PCR reaction. Primer length is 18-30 bp with Tm of 58-62° C. Each primer includes part of the standard Illumina TrueSeq adaptors (25 bp), not including the Index. In addition to the target specific sequence, the left primers have the sequence TCCCTACACGACGCTCTTCCGATCT (SEQ ID NO: 1) and right primers have the sequence AGTTCAGACGTGTGCTCTTCCGATC (SEQ ID NO: 2). These sequences were added to the 5′ ends of the primers.

All primers were mixed in the same reaction tube and the PCR was prepared using QIAGEN Multiplex PCR Kit according to the manufacturer's instructions. The reaction conditions of first PCR were: 95° C. for 15 min, followed by 30 cycles of 95° C. for 30 sec., 57° C. for 3 min and 72° C. for 1.5 min, followed by 10 min. at 68° C.

Exonuclease step: Products from the 1st step PCR were treated with Exonuclease I (ThermoScientific) for primer removal according to the manufacturer's instructions. 2nd Step PCR: Cleaned PCR products from the 1st PCR step were amplified using one pair of truncated TrueSeq universal adaptor primers including index (Illumina), allowing the mixing of samples from different individuals. One pair of index primers was added to each 1st PCR reaction, to differentially label the products from each template DNA sample. PCR was prepared using 2× PCRBIO HS Taq Mix Red Kit (PCR Biosystems) according to manufacturer's instructions. The truncated universal adaptor primers (from 5′ to 3′) were as follows indicates variable indexes): Forward: AATGATACGGCGACCACCGAGATCTACACTCTTTCCCTACACGACGCTC (SEQ ID NO: 7); Reverse index primer: CAAGCAGAAGACGGCATACGAGA GTGACTGGAGTTCAGACGTGTG CTC (SEQ ID NO: 8). Alternatively, no index can be used, and the reverse primer is CAAGCAGAAGACGGCATACGAGATGTGACTGGAGTTCAGACGTGTGCTC (SEQ ID NO: 9).

The reaction conditions of the 2nd step PCR were: 95° C. 2 min for activation enzyme, followed by 15 cycles of 95° C. 30 sec., 59° C. 1.5 min., 72° C. 30 sec, followed by 10 min. at 72° C. The products from multiple 2nd PCR reactions can be combined to one tube (since each 1st PCR product cocktail is differentially indexed). PCR products were run on 3% agarose gels with ethidium bromide staining and extracted by Zymo GEL Recovery kit.

### Next Generation Sequencing

Sequencing was performed on PCR products using MiSeq Reagent Kit v2 (MiSeq, Illumina method) or NextSeq 500/550 v2 sequencing reagent kits. Sequenced reads were separated by barcode, aligned to the target sequence and analyzed using custom scripts written and implemented in R. Reads were quality filtered based on Illumina quality scores. Reads were identified by having at least 80% similarity to target sequences and containing all the expected CpGs in the sequence. CpGs were considered methylated if “CG” was read and considered unmethylated if “TG” was read. Proper bisulfite conversion was assessed by analyzing methylation of non-CpG cytosines. Fraction of molecules containing fully unmethylated DNA fragment was determined. The fraction obtained was multiplied by the concentration of cfDNA measured in each sample, to obtain the concentration of tissue specific derived DNA circulating in the blood of each donor.

### Example 1: Design of Primers for 2 Step PCR

Assessment of the methylation status of specific loci, via bisulfite treatment followed by PCR and sequencing, allows for identification of the tissue of origin of cell-free circulating DNA (cfDNA). However, amplifying more than one PCR product in the same reaction is difficult. In addition, the amount of available DNA is often limiting in the setting of cell-free circulating DNA and other applications; therefore, determining the methylation status of multiple markers using the same sample (e.g. small volume plasma sample) is challenging.

The plasma of healthy individuals contains approximately 1,000 genome equivalents (GEq) of total cfDNA per mL, rendering the detection of rare cfDNA populations challenging. Bisulfite treatment deaminates unmethylated cytosines to uracils while leaving methylated cytosines intact, allowing for the detection of methylation patterns; however, bisulfite destroys ˜80% of DNA molecules. Hence, when starting with 1 ml of plasma, a cell type contributing 1% to the total circulating cfDNA will be present at approximately 2 GEq after bisulfite conversion, a level which may not be detected consistently by PCR for any given locus. The sensitivity of cfDNA assays can theoretically be increased by using a larger volume of blood, or by simultaneously assessing multiple markers for the cell type of interest.

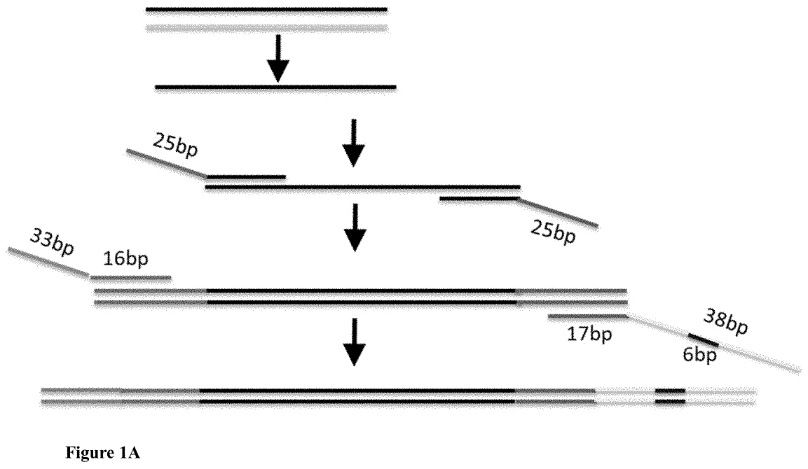

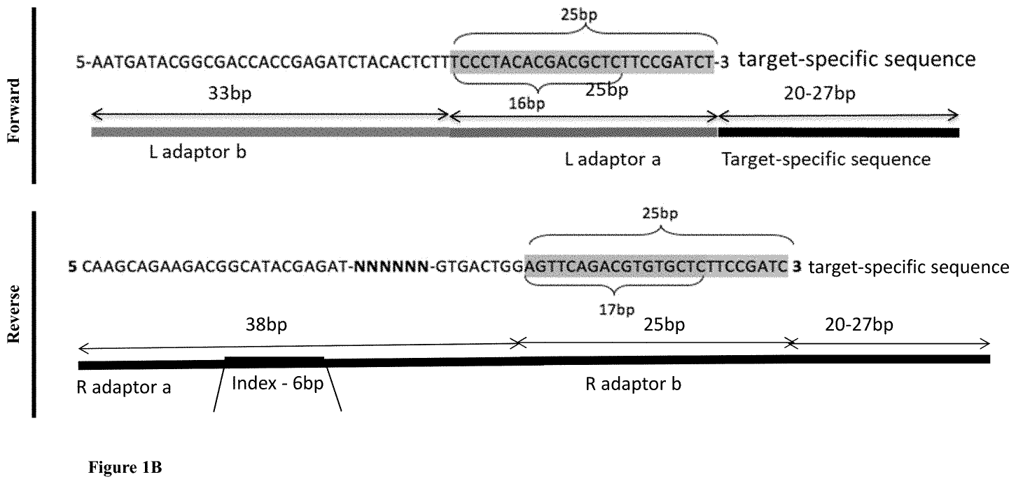

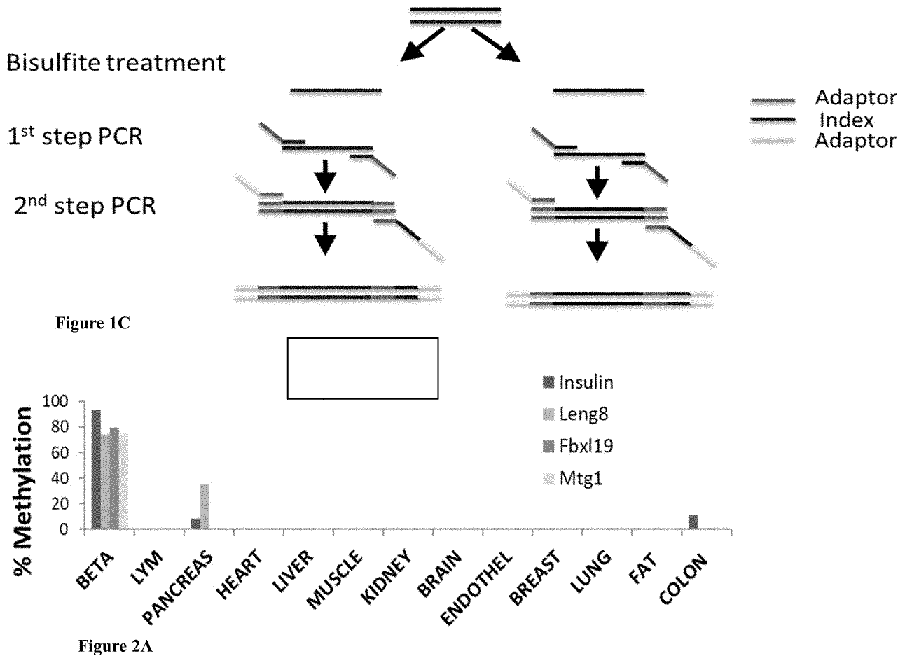

In order to solve this problem, two sets of primer pairs were designed that could be used for PCRing multiple loci from a single sample, specifically a bisulfite-treated human cfDNA sample. The first primer pair is specific to a given loci. Forward and reverse primers of length 20-27 nucleotides are selected specificity for the given methylation locus. The primers are designed without the inclusion of cytosines (which may be converted to thymine during bisulfite treatment), which generally results in a lower Tm. An adapter is then added to the 5′ end of each primer. The forward adapter and reverse adapter are both 25 nucleotides and are taken from the 3′ ends of the TruSeq Universal Adapter. This results in primers of between 45-52 nucleotides in length that are specific to a target sequence but will produce an overhang after the first cycle of PCR (FIG. 1A). Multiple sets of these primers can be used simultaneously in the first round of PCR (20 cycles) to amplify several loci at once.

The second PCR step uses a pair of universal primers, derived from the TruSeq Universal Adapter to further amplify all of the products from the first PCR step. These universal primers have 3′ ends that are the same as the 5′ ends of the first primer pair. However, the 3′ ends do not comprise the entire 25 nucleotides that were added to the first primer pairs, but rather only a portion. Specifically, the forward primer has a 3′ end that is the same as the 5′-most 16 nucleotides of the 1^{st} step forward primers, and the reverse primer has a 3′ end that is the same as the 5′-most 17 nucleotides of the 1^{st} step reverse primers (FIG. 1A). As such the universal primers overlap with only 60-70% of the adapter region. The rest of the sequence of the universal 2^{nd} step primers is the same as the 5′ ends of the TruSeq Universal Adapter (FIG. 1B).

Bisulfite converted DNA can be sequenced following use of these primers in a 2 step PCR that yields sequencing-ready products. Further, the process allows for assaying both sense and anti-sense strands of the converted DNA (FIG. 1C).

These specific sizes for the overlap and overhang of the primers were selected to improved amplification and fidelity. A shorter overlap would decrease fidelity and result in non-specific binding. Further, because the first 12 nucleotides of both the forward and reverse adapters are identical shorter overlaps would have resulted in binding from both sides. Longer overlaps resulted in first primers that were to long as the overlap must be added to an already full-length primer. Overlap of 12-17 nucleotides were found to be ideal

### Example 2: 2-Step Multiplex PCR Increases Assay Sensitivity

As a proof of principle 4 beta-cell methylation loci were assayed together using the primers of the invention. The loci specific regions of the primers are presented in Table 1.

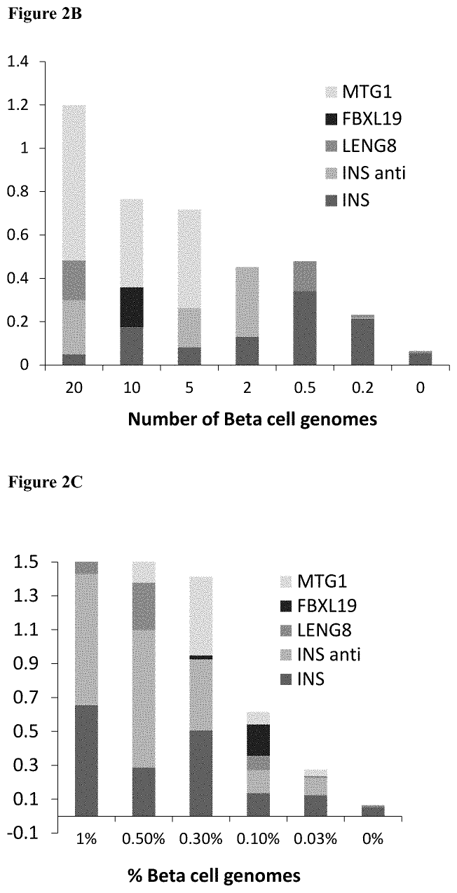

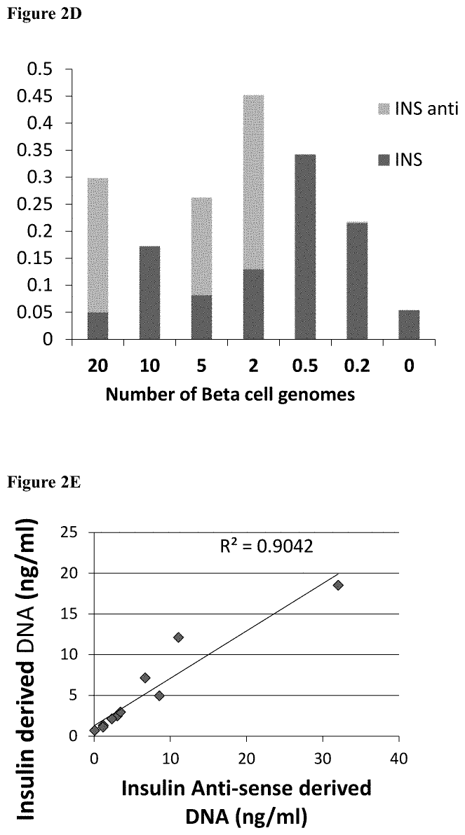

All 4 loci were found to be highly methylated (over 70%) in beta cells, and unmethylated in nearly every other tissue (FIG. 2A). By using methylation of these loci as a signal of beta cell DNA, the relative amount of beta cell DNA present in blood could be evaluated. Beta cell DNA was spiked into healthy blood and 2-step PCR was performed for these 4 loci, and as a proof of principle the anti-sense strand of the Insulin locus. The beta cell DNA could be detected when even as little as half a genome was added (FIG. 2B), or even when the spiked in DNA was only 0.03% of the total DNA present (FIG. 2C) attesting to the sensitivity of the assay.

Importantly, this method allows for amplification of both the sense and anti-sense strand of a given loci (FIG. 2D). This provides increased information of strand-specific methylation and can help confirm the presence of a give loci with high assurance (FIG. 2E). This is especially important when analyzing bisulfate treated molecules as the decrease in diversity of sequence can make identification of the loci being probed harder to evaluate.

### Example 3: 2-Step Multiplex PCR with Samples from Patients

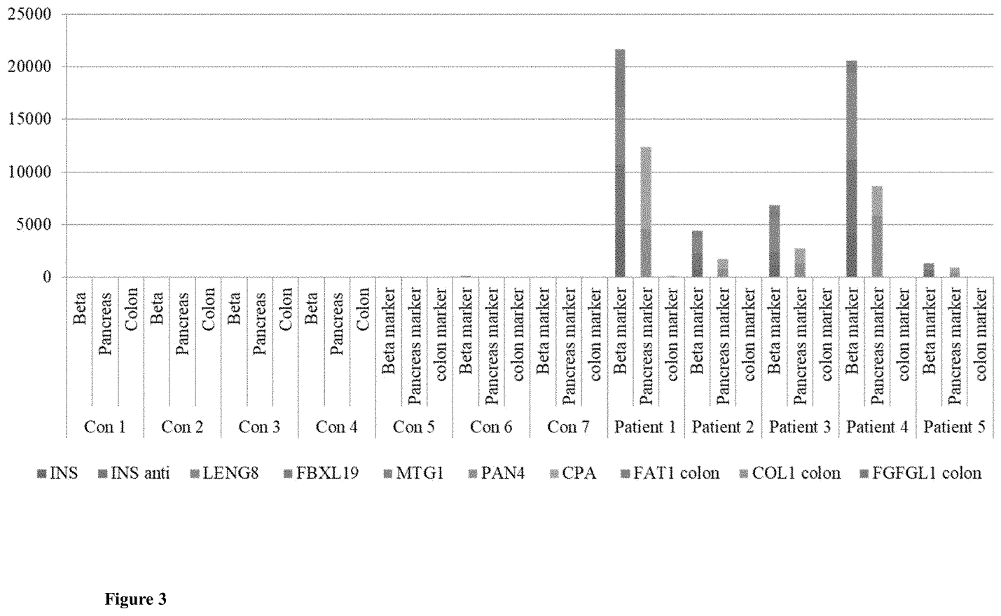

The 2-step PCR was next tested with bisulfite treated cfDNA from patient blood samples. Blood was gathered from five recent recipients of islet transplantation and seven control subject who had not undergone transplantation. cfDNA was isolated and bisulfite conversion was performed. Each subject's sample was PCRed with ten methylation specific primer pairs: 5 beta cell specific markers already described (Insulin sense, Insulin anti-sense, LENG8, FBXL19, and MTG1), two exocrine pancreases markers (PAN4 and CPA) and three colon markers (FAT1, COL1 and FGFGL1). Beta cell markers were the most highly detected and were present in all five patients (FIG. 3). Exocrine pancreas markers were also detected in all five patients, while colon markers were not detected.

### Example 4: High Fidelity Even with Low Sample Amounts

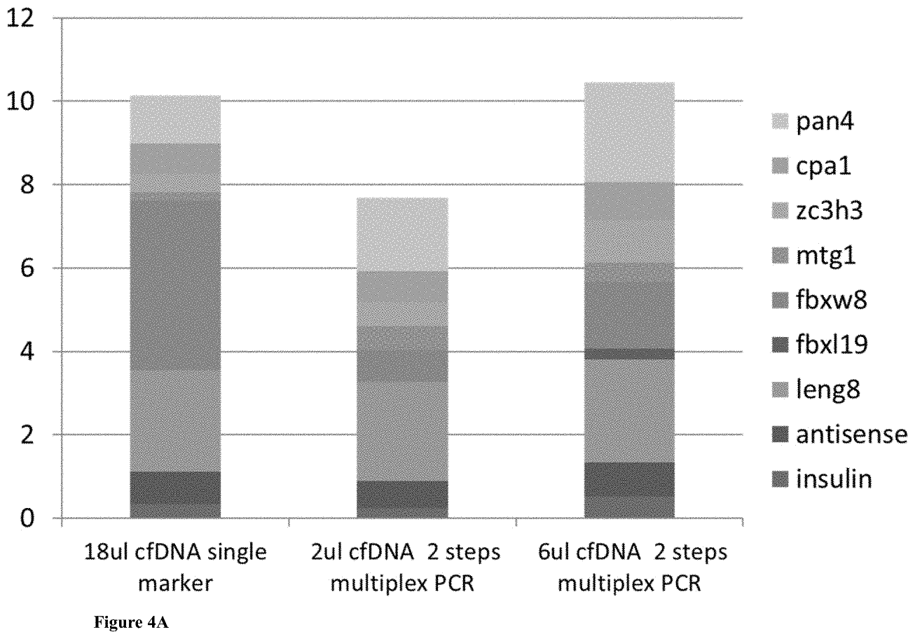

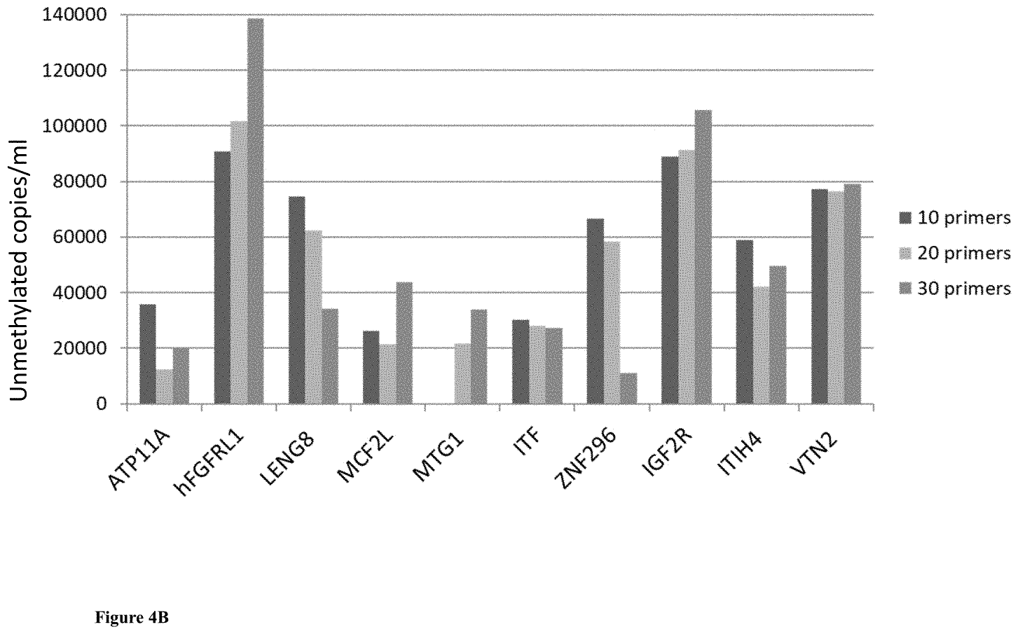

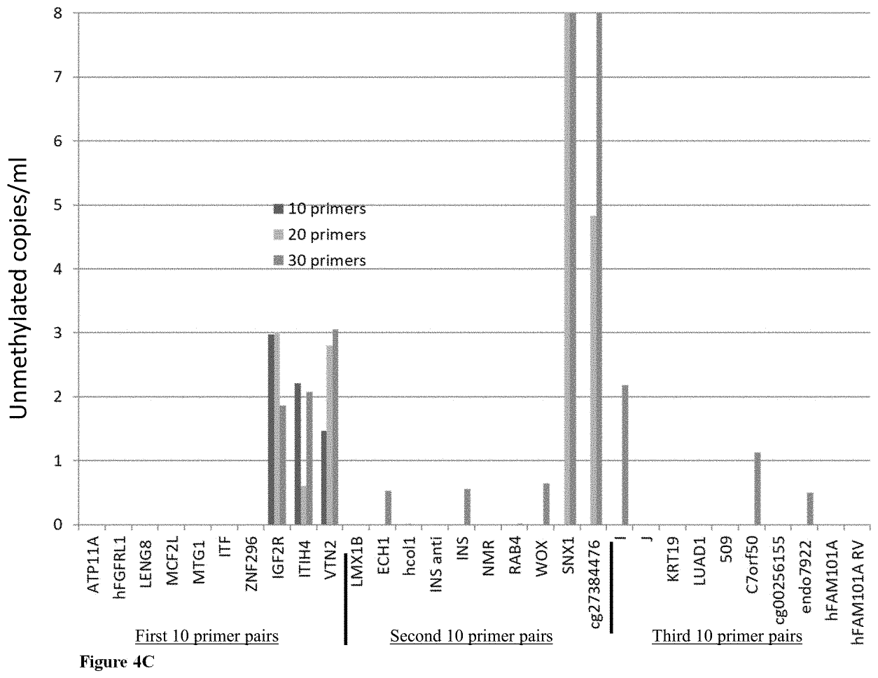

Next, a sample from a transplant recipient was taken and the needed abundance of starting material was assayed. Nine pancreas related loci were probed. First 2 ul of bisulfite converted cfDNA was used for each PCR separately, for a total of 18 ul needed (FIG. 4A, left column). Then multiplex PCR was performed with only 6 or 2 ul total for the entire reaction (FIG. 4A, right and middle columns respectively). All markers were detected even when only 2 ul of starting material were used and as robust if not greater expression was observed when 6 ul total was used (a 66.7% reduction in starting material).

Having already demonstrated that 10 primer pairs could be used on a single sample, the number of primers was increased to 20 or 30 and the fidelity and sensitivity of the multiplex PCR was evaluated. First, 10 pairs of primers were used for multiplex PCR on a mix of cfDNA extracted from several healthy blood samples. Of those 10 primer pairs, 5 were for cell types that generally appear even in healthy blood samples (3 liver-specific loci, 1 leukocyte-specific loci and 1 endothelial-specific loci) (FIG. 4B, left bars), the other 5 were irrelevant loci. Then the same PCR was performed, but primers for 10 irrelevant loci were added (FIG. 4B, middle bars), and then 10 more irrelevant loci were added (FIG. 4B, right bars). PCR results were consistent regardless of how many primer pairs were used. When the experiment was repeated on the same mix of cfDNA similar results were observed (FIG. 4C). In addition, consistent PCR results were also found in the 20 primers set and in the 30 primers set for one leukocytes (SNX1) and for one endothelial loci (cg27384476) regardless of how many primer pairs were used.

### Example 5: Expansion of the Multiplex Assay for Beta-Cell DNA

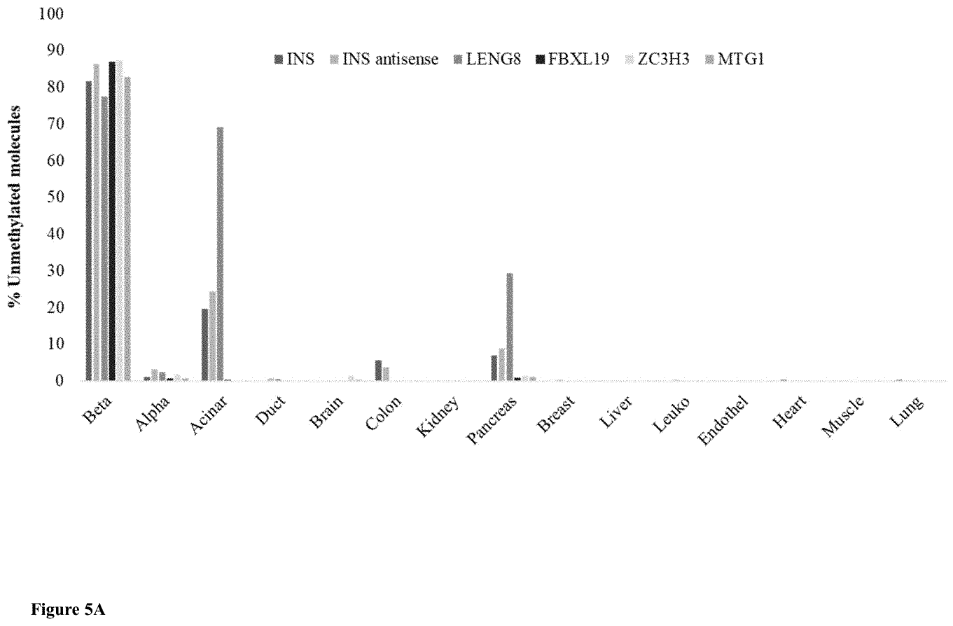

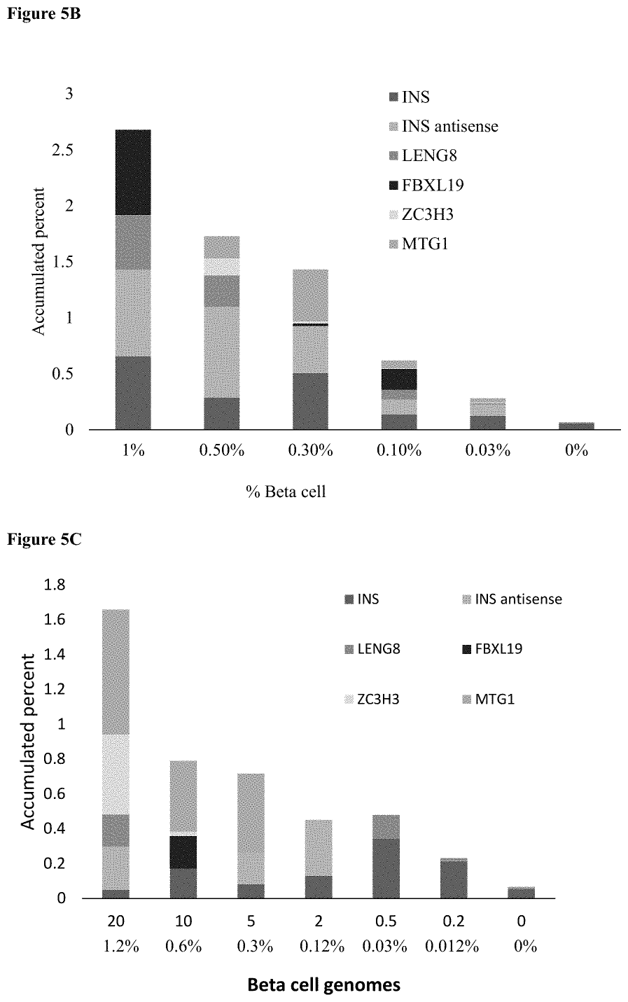

To validate the specificity of the β-cell-specific marker cocktail, bisulfate-treated genomic DNA from a panel of tissues was amplified and sequenced. In contrast to previous studies which scored the methylation status of individual CpG sites from Illumina methylation arrays, this analysis reported the methylation status of all CpG sites in the amplified loci. It is known that scoring only molecules that are unmethylated in all CpGs greatly increases assay specificity by reducing noise level in unrelated tissues. All six markers (Insulin, Insulin antisense, LENG8, FBXL19, MTG1 and ZC3H3) were completely unmethylated in >70% of sorted β-cells (FIG. 5A), while occasional methylation at 1-2 CpGs per molecule was detected from the remaining 30% of β-cells (not shown). There were no fully unmethylated molecules in other tissues examined, with two exceptions: 1) insulin and a sequence adjacent to the Leng8 gene were unmethylated in 30-40% of pancreatic acinar cells (and, accordingly, also in unsorted human pancreas); and 2) insulin was unmethylated in 11% of DNA molecules from intestinal epithelial cells (FIG. 5A).

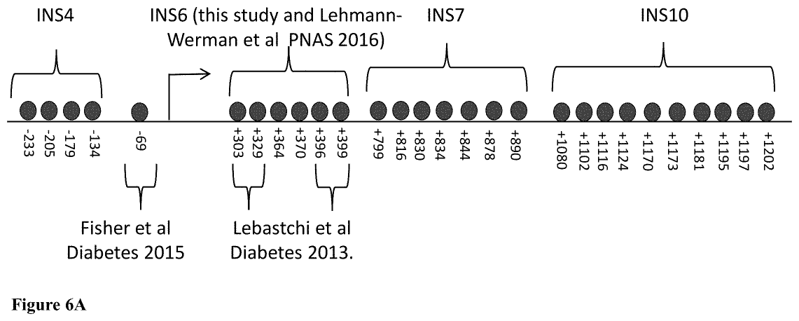

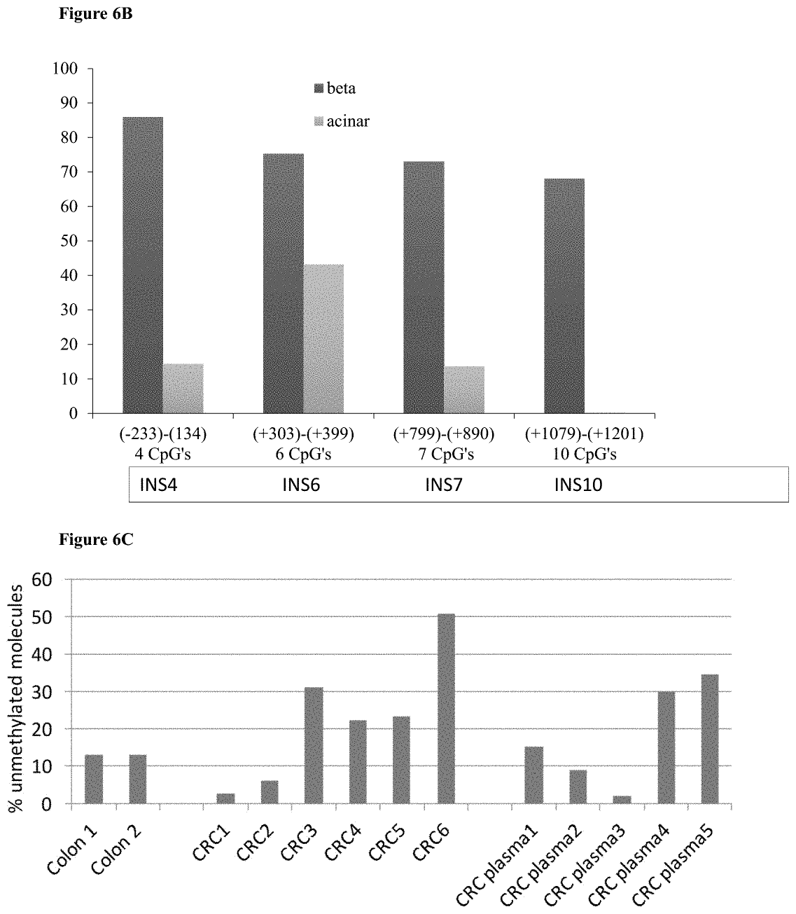

Prompted by these unexpected findings, a systematic analysis of the methylation pattern along the insulin gene was performed and several areas that were demethylated in a fraction of cells in the exocrine pancreas, as well as a region that was unmethylated exclusively in β-cells (1058-1222 bp downstream to the INS transcription start site (TSS), SEQ ID NO: 188) were identified (FIG. 6A-B). In addition, it was verified that the insulin promoter was demethylated in a fraction of intestinal cells within human intestinal samples obtained from normal control subjects, colorectal cancer biopsies, and even cfDNA obtained from patients with colorectal cancer (FIG. 6C). These findings cast doubt on the reliability and precision of INS DNA alone, except in this particular region, as a circulating methylation marker of β-cell-derived cfDNA and suggest the existence of previously unappreciated epigenetic heterogeneity in the exocrine pancreas and intestine. Nonetheless, the data support the use of a multiplex assay, combining insulin and additional β-cell specific markers, to detect β-cell cfDNA.

To determine assay sensitivity, mixtures of leukocyte and β-cell derived DNA in known proportions were prepared. β-cell DNA was robustly identified when 10 GEq of β-cell DNA (60 pg) comprised 1%, 0.5%, 0.3%, 0.1% and 0.03% of the mixture, equivalent to sensitivity of gold standards in the field of cancer liquid biopsies (FIG. 5B). Similar findings were obtained when different absolute amounts of β-cell DNA were included in the mixtures. Even when only 0.2 β-cell GEq was present, at least one of the markers in the cocktail provided a signal distinct from the baseline level of pure leukocyte DNA (FIG. 5C). It was thus concluded that the new multiplex assay for β-cell DNA enhances sensitivity and specificity compared with methods based on methylation of the insulin gene alone.

### Example 6: Low Levels of β-Cell cfDNA Throughout the Lifespan of Healthy Individuals

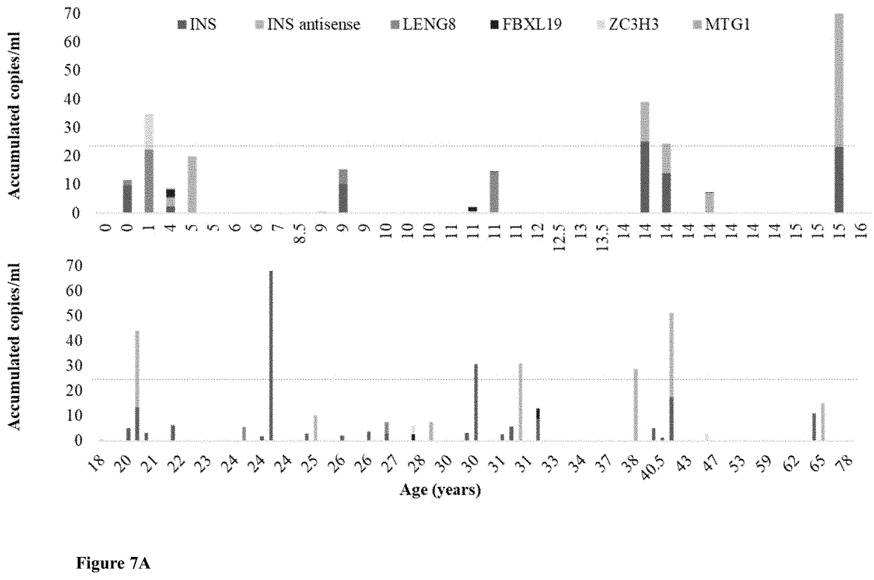

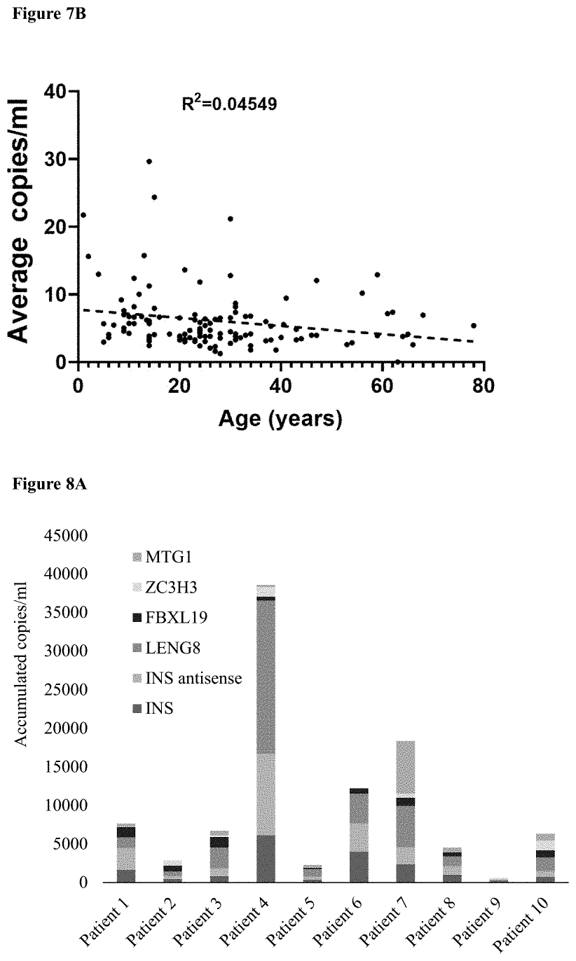

Next cfDNA was isolated from at least 2 mL plasma (representing approximately 400 GEq following bisulfite treatment) from each of 121 healthy subjects aged 4-78 years and β-cell derived cfDNA was measured using the six-marker multiplex assay. FIG. 7A shows the level of signal for each marker from each sample evaluated. Consistent with the quiescent nature of β-cells during postnatal life, β-cell-derived cfDNA was extremely rare in plasma from healthy subjects. In most positive samples, only one or two of the six markers were detected. To assess the concentration of β-cell GEq from cfDNA, the average signal from all six markers tested was calculated, and it was found that healthy individuals had on average just one β-cell-derived GEq/mL plasma. Subjects were then binned into three age groups (children (4-10 years; n=16), adolescents (11-18 years; n=20), and adults (18-78 years; n=85)) and it was determined that control subjects to have comparable minimal levels of β-cell cfDNA in plasma throughout lifespan (FIG. 7B).

### Example 7: Elevated Levels of β-Cell cfDNA Immediately after Islet Transplantation

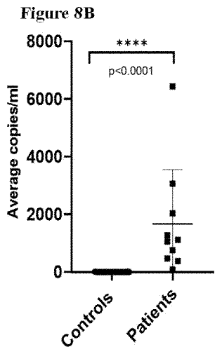

Patients transplanted with cadaveric or auto-transplanted islets contain massive amounts of β-cell-derived cfDNA in the hours that follow islet infusion. This was determined based on assays measuring demethylated insulin DNA alone, as well as upon deconvolution of the entire plasma methylome. As expected, all six β-cell methylation markers (FIG. 8A) were significantly elevated in the plasma of 10 islet transplant recipients, one-hour post-transplant, as compared to healthy controls (FIG. 8B), further validating performance of the multiplex assay. The presence of β-cell derived cfDNA in the islet transplant setting is interpreted to reflect β-cell death, with signal representing DNA from β-cells that died before, during, and after transplantation.

### Example 8: β-Cell cfDNA in a Patient with Congenital Hyperinsulinism of Infancy (CHI)

Evidence from rare surgical specimens of children with CHI suggests that, in addition to hypersecretion of insulin, the disease involves an increased rate of β-cell turnover. Histologic sections of pancreases resected from patients with CHI due to inactivating mutations in genes encoding the β-cell Katp channel show increased numbers of both proliferating and apoptotic β-cells when compared to age matched controls. This finding is consistent with the clinical observation of progressive improvement in hypoglycemia with the development of diabetes in some CHI patients after conservative (non-surgical) treatment. The mechanism of this clinical progression is not known since there are no biomarkers of human CHI dynamics and liquid biopsies have not been reported.

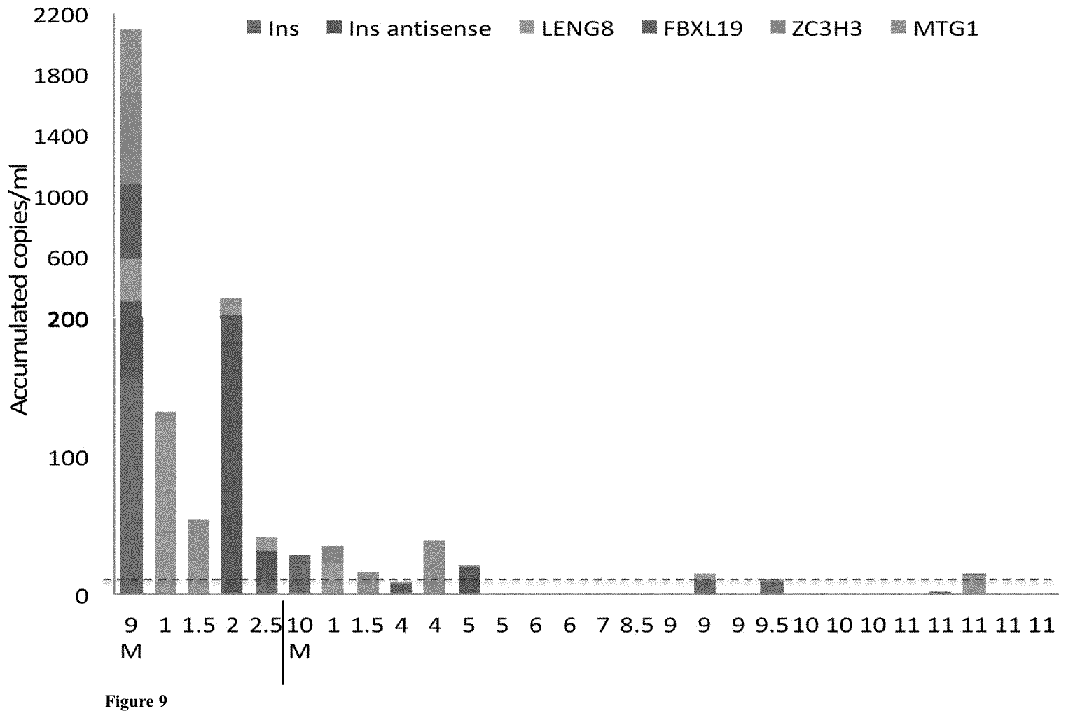

Five longitudinal plasma samples from a child with CHI spanning over a period of 21 months (age 9 months to 2.5 years) were obtained. This child carried a de-novo (i.e. both parents were negative) missense dominant ABCC8 mutation, c.4453G>A causing a change in the protein p.Glyl485Arg (p.G1485R). This particular mutation has previously been reported. Importantly, dominantly acting ABCC8 mutations have been reported to confer risk of diabetes in adulthood. Strikingly, all five samples showed a β-cell cfDNA signal (348-6.8 GEq/mL), derived from multiple markers, that was clearly above baseline levels recorded in 23 age-matched controls from the same clinic (FIG. 9). A later sample taken at age 3.5 years was negative (not shown). These findings support the ability of the multiplex cocktail to detect non-malignant β-cell death occurring in the native pancreas environment with various combinations of the six β-cell markers driving the signal detected in each sample, pointing to the added sensitivity provided with the inclusion of multiple methylation markers. Beyond serving as validation for this methodology, these finding suggest that liquid biopsies can provide insights into CHI disease progression.

### Example 9: Multiple Unique Methylation Markers for Various Cells/Tissues Increase Assay Specificity and Sensitivity

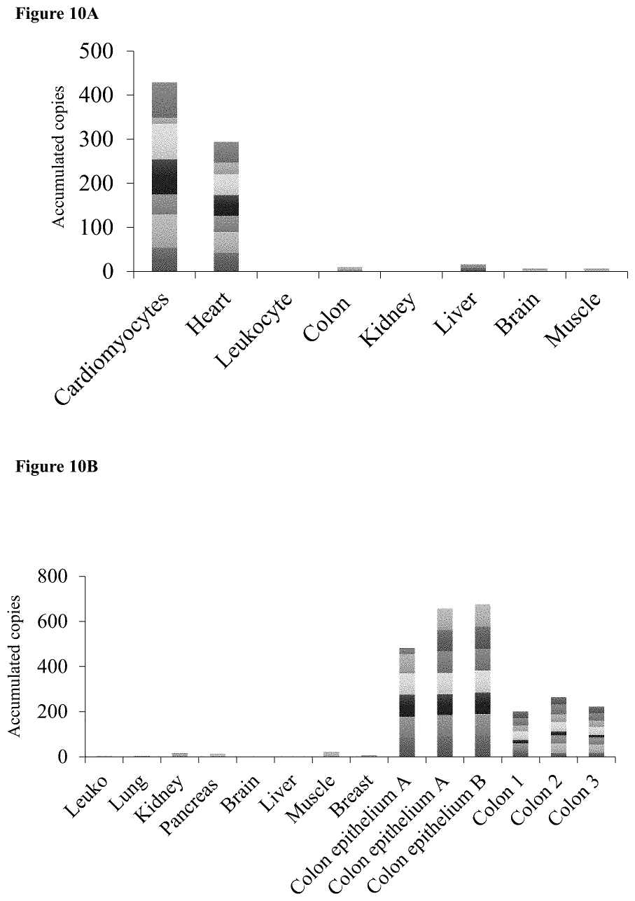

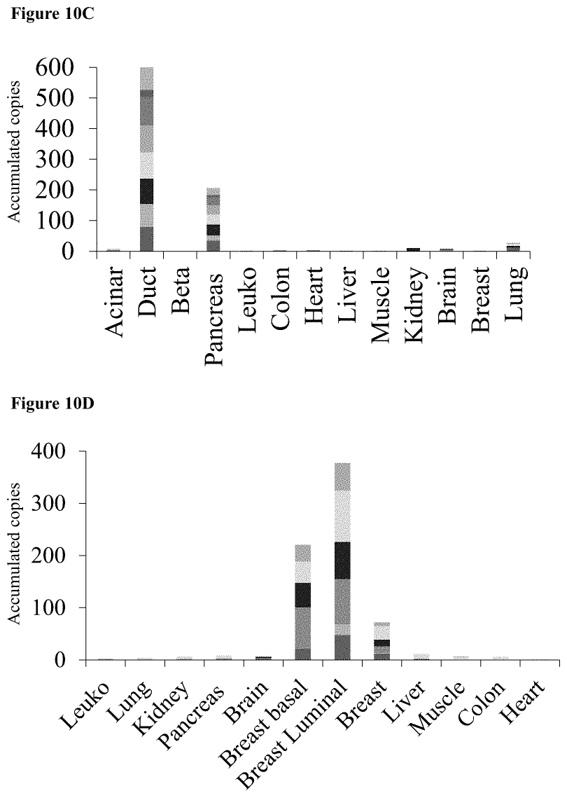

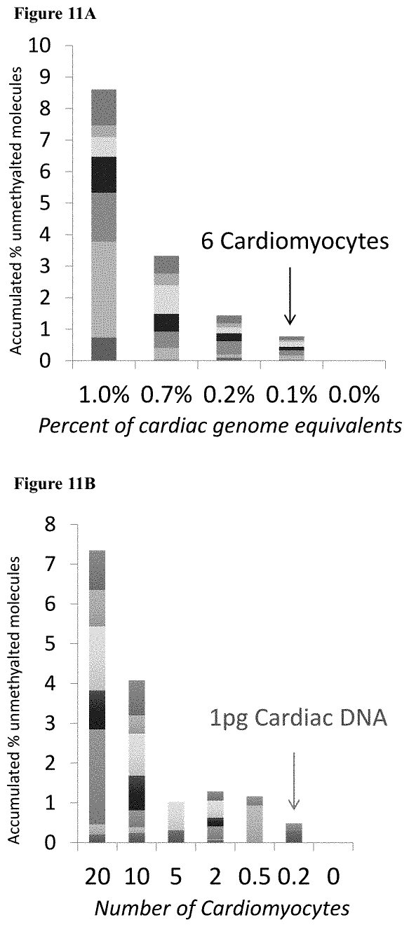

The use of multiple markers per cell type or tissue to enhance the efficacy of the multiplex PCR was tested. First, seven markers for cardiomyocytes were tested together for their ability to specifically identify heart cells. As can be seen in FIG. 10A, though any one of the markers might be detected in other tissues, the combination of two or more markers allows for the specific identification of only heart cells.

Similar tests were performed with seven markers for colon cells (FIG. 10B), 8 markers for pancreatic duct cells (FIG. 10C) and six markers for breast cells (10D). Though one marker can often be found as expressed in other tissues, the combination of two or more markers resulted in a highly specific test detecting only expected tissues/cells. With the addition of each marker beyond two, the assay became even more reliable and specific.

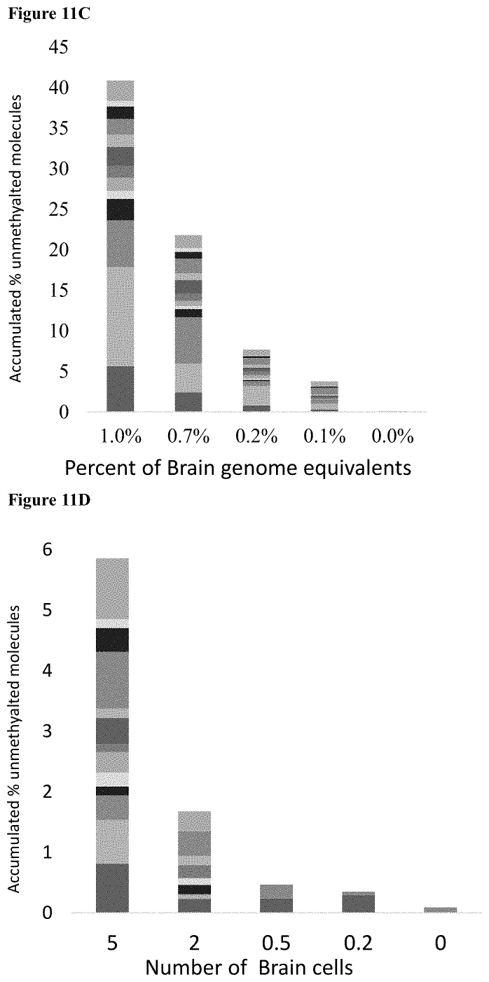

It was also found that the use of multiple markers enhances the sensitivity of the assay. Cardiomyocyte DNA was mixed with leukocyte DNA in different proportions and the ability to still detect cardiac markers was tested. First, 20 genome equivalents of cardiac cell DNA was diluted in various amounts of whole blood DNA. Even at 0.1% genome equivalents (equal to DNA of 6 cardiomyocytes) the cardiomyocyte DNA was readily detectable when 7 markers were measured together (FIG. 11A). Similarly, with even as little as 1 picogram of cardiac DNA, the equivalent of 1/5 of a cell, diluted in 10 ng of blood DNA, the seven markers were capable of detecting cardiac DNA (FIG. 11B). The same experiment was performed using brain cell DNA (from neurons, oligodendrocytes and astrocytes) spiked into blood DNA. Due to the diverse number of cell types that make up the brain cell DNA, 12 markers were used instead of seven (CG0978, WB1, UBE, NMR, TAF8, ZFP, 509, ITF, SLC, ZNF238, WOX and AST1). As was observed for cardiomyocytes, even 0.1% genome equivalents (FIG. 11C) or 1 pg/a fifth of a cell (FIG. 11D) could be detected.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A primer set comprising:
a. a first primer comprising a 3′ region of homology to a 5′ segment of a target nucleic acid molecule and a 5′ first adapter sequence wherein said first adapter sequence comprises a 5′ first and 3′ second region and wherein said first region is between 50% and 99% of the first adapter sequence; and
b. a second primer comprising a 3′ region identical to said first region of said first adapter sequence and a 5′ second adapter sequence.

2. The primer set of claim 1, further comprising a third primer comprising a 3′ region that is a reverse compliment to a 3′ segment of said target nucleic acid molecule and suitable for amplifying said target molecule in combination with said first primer and a fourth primer, wherein said third primer further comprises a 5′ third adapter sequence wherein said third adapter sequence comprises a first and second region and wherein said fourth primer comprises a 3′ region identical to said first region of said third adapter sequence and a 5′ fourth adapter sequence.

3. (canceled)

4. The primer set of claim 2, wherein said first region of said third adapter sequence is between 50% and 99% of the third adapter sequence.

5. The primer set of claim 3, wherein said second region of said first adapter sequence and said second region of said third adapter sequence are at least 85% identical.

6. The primer set of claim 1, wherein said 3′ region of said second primer and/or said fourth primer is less than 35% of said second and/or fourth primer.

7. The primer set of claim 1, wherein said first region of said first adapter is between 14 and 19 nucleotides, said second region of said first adapter is between 7 to 11 nucleotides or both.

8. (canceled)

9. The primer set of claim 2, wherein said second primer, said fourth primer or both further comprises a barcode 5′ of said 3′ region, optionally wherein said third adapter sequence comprises the sequence AGTTCAGACGTGTGCTCTTCCGATC (SEQ ID NO: 2).

10. The primer set of claim 14, wherein said first adapter sequence comprises the sequence TCCCTACACGACGCTCTTCCGATCT (SEQ ID NO: 1), said second region of said first adapter and/or said second region of said third adapter comprises the sequence TTCCGATC (SEQ ID NO: 3), or both.

11. (canceled)

12. (canceled)

13. A kit, comprising at least two primer sets of claim 1, wherein each first primer comprises a 3′ region of homology to a different target nucleic acid molecule and
a. wherein the second adapter sequence is a universal sequence shared by all second primers;
b. wherein the fourth adapter sequence is a universal sequence shared by all fourth primers, or
c. both a and b.

14. A method of polymerase chain reaction (PCR), the method comprising:
a. providing a sample comprising a target nucleic acid molecule;
b. performing a first PCR reaction with said target nucleic acid molecule and a first primer of a primer set of claim 1 to produce a first adapter labeled target nucleic acid hybrid molecule; and
c. performing a second PCR reaction with said hybrid molecule and a second primer of a primer set of claim 1 to produce a first and second adapter labeled target nucleic acid hybrid molecule.

15. A method of generating a sequencing library, the method comprising performing the method of claim 14, wherein:
a. said providing is providing a sample comprising at least two target nucleic acid molecules;
b. said performing a first PCR reaction is with said at least two target nucleic acid molecules and at least two first primers of said primer set to produce first adapter labeled target nucleic acid hybrid molecules, wherein said at least two first primers are homologous to different target nucleic acid molecules and comprise identical first regions of a first adapter; and
c. said performing a second PCR reaction is with said hybrid molecules and a second primer of said primer set to produce first and second adapter labeled target nucleic acid hybrid molecules;
thereby generating a sequencing library.

16. The method of claim 15, wherein said sample comprises cell free DNA (cfDNA).

17. The method of claim 14, wherein said sample comprises bisulfite converted nucleic acids.

18. The method of claim 17, wherein said at least two target nucleic acid molecules are a first and second strand of a target double stranded bisulfite converted DNA.

19. The method of claim 15, further comprising sequencing said first and second adapter labeled nucleic acids of the sequencing library.

20. A method of determining the cell type of origin of cfDNA, the method comprising:
a. providing a sample comprising at least two target nucleic acid molecules, wherein said at least two target nucleic acid molecules are cfDNA, that comprise at least one cell type-specific methylation/unmethylated site and wherein said at least two target nucleic acid molecules of cfDNA have undergone bisulfite conversion;
b. performing a first PCR reaction with said at least two target nucleic acid molecules and at least two first primers to produce first adapter labeled target nucleic acid hybrid molecules, wherein each of said first primers comprises
i. a 3′ region of homology to a 5′ segment of one of said at least two target nucleic acid molecules; and
ii. a 5′ first adapter sequence common to all first primers wherein said first adapter sequence comprises a 5′ first and 3′ second region;
c. performing a second PCR reaction with said first primer labeled hybrid molecules and at least two second primers to produce first and second adapter labeled target nucleic acid hybrid molecules, wherein each of said second primers comprises
i. a 3′ region identical to said first region of said first adapter sequence; and
ii. a 5′ second adapter sequence;
d. sequencing said first and second adapter labeled target nucleic acid molecules; and
e. determining a methylation status of said methylation/unmethylated site according to the base sequenced at said methylation/unmethylated site;
wherein the presence of a methylation mark or lack of a methylation mark that is cell type-specific indicates that said cfDNA originates from said cell type, thereby determining the cell type of origin of said cfDNA.

21. The method of claim 20, wherein said first PCR is performed with
a. at least 3 first primers that each comprise a region of homology to a target nucleic acid molecule with a cell type-specific methylation/unmethylated site for a different cell type;
b. at least 3 first primers that each comprise a region of homology to a target nucleic acid molecule with a different cell type-specific methylation/unmethylated site for the same cell type;
c. a first first primer with homology to a forward strand of a nucleic acid molecule with a cell type-specific methylation/unmethylated site and a second first primer with homology to a reverse strand of the same nucleic acid molecule with a cell-type specific methylation/unmethylated site; or
d. a combination thereof.

22. (canceled)

23. (canceled)

24. (canceled)

25. The method of claim 20, wherein said sample is from a subject and said determining a cell type of origin comprises detecting cell death of cells of said cell type of origin within said subject, optionally wherein said cell type is β-cells, and said method is for detecting β-cells death within said subject.

26. (canceled)

27. The method of claim 15, wherein said first PCR reaction comprises 15 to 25 cycles and wherein said PCR reaction is a gradient reaction wherein the annealing temperature increases during said first PCR reaction.

28. A method of detecting beta cell cfDNA in a sample, the method comprising:
a. receiving a sample comprising cfDNA;
b. detecting in said sample a DNA sequence of a region of an insulin (INS) gene, wherein said region is between nucleotides 1058-1222 downstream of an INS transcriptional start site and comprises at least one cytosine base selected from cytosine 1080, 1102, 1116, 1124, 1170, 1173, 1181, 1195, 1197 and 1202; and
c. determining a methylation status of said at least one cytosine base, wherein absence of methylation on said at least one cytosine base indicates the presence of beta cell cfDNA;
thereby detecting beta cell cfDNA in a sample.
